(19) United States
(12) Patent Application Publication
(10) Pub. No.: US 2023/0034913 A1
(43) Pub. Date: Feb. 2, 2023

(54) METHODS, SYSTEMS, AND COMPOSITIONS FOR MAINTAINING FUNCTIONING DRAINAGE BLEBS ASSOCIATED WITH FOREIGN BODIES

(71) Applicant: RADIANCE THERAPEUTICS, INC., Tucson, AZ (US)

(72) Inventor: Laurence J. Marsteller, Tucson, AZ (US)

(21) Appl. No.: 17/861,069

(22) Filed: Jul. 8, 2022

Related U.S. Application Data
(63) Continuation-in-part of application No. PCT/US2021/012694, filed on Jan. 8, 2021.
Application No. PCT/US2021/012694 is a continuation-in-part of application No. PCT/US2021/012744, filed on Jan. 8, 2021.
Application No. PCT/US2021/012744 is a continuation-in-part of application No. 17/694,366, filed on Mar. 14, 2022.
Application No. 17/694,366 is a continuation-in-part of application No. 16/698,676, filed on Nov. 27, 2019, now Pat. No. 11,273,325.
Application No. 17/694,366 is a continuation-in-part of application No. PCT/US2021/012694, filed on Jan. 8, 2021.
Application No. PCT/US2021/012694 is a continuation-in-part of application No. PCT/US2021/012744, filed on Jan. 8, 2021.
Application No. PCT/US2021/012744 is a continuation-in-part of application No. 17/676,711, filed on Feb. 21, 2022.
Application No. 17/676,711 is a continuation-in-part of application No. PCT/US2020/047235, filed on Aug. 20, 2020.
Application No. 17/676,711 is a continuation-in-part of application No. PCT/US2021/012694, filed on Jan. 8, 2021.
Application No. PCT/US2021/012694 is a continuation-in-part of application No. PCT/US2021/012744, filed on Jan. 8, 2021.
Application No. PCT/US2021/012744 is a continuation-in-part of application No. 16/698,676, filed on Nov. 27, 2019, now Pat. No. 11,273,325.
Application No. 17/694,366 is a continuation-in-part of application No. 17/676,711, filed on Feb. 21, 2022.
Application No. 17/676,711 is a continuation-in-part of application No. PCT/US2021/064141, filed on Dec. 17, 2021.
Application No. PCT/US2021/064141 is a continuation-in-part of application No. PCT/US2021/012744, filed on Jan. 8, 2021.
Application No. PCT/US2021/012744 is a continuation-in-part of application No. PCT/US2021/012694, filed on Jan. 8, 2021.
Application No. 17/694,366 is a continuation-in-part of application No. PCT/US2021/064141, filed on Dec. 17, 2021.
Application No. 17/676,711 is a continuation-in-part of application No. PCT/US2021/064141, filed on Dec. 17, 2021.
Application No. PCT/US2021/064141 is a continuation-in-part of application No. PCT/US2021/064190, filed on Dec. 17, 2021.
Application No. PCT/US2021/064190 is a continuation-in-part of application No. PCT/US2021/012744, filed on Jan. 8, 2021.
Application No. PCT/US2021/012744 is a continuation-in-part of application No. PCT/US2021/012694, filed on Jan. 8, 2021.
Application No. 17/694,366 is a continuation-in-part of application No. PCT/US2021/064190, filed on Dec. 17, 2021.
Application No. 17/676,711 is a continuation-in-part of application No. PCT/US2021/064190, filed on Dec. 17, 2021.
Application No. PCT/US2021/064190 is a continuation-in-part of application No. 17/782,940, filed on Jun. 6, 2022.
Application No. 17/782,940 is a continuation-in-part of application No. 16/584,737, filed on Sep. 26, 2019.
Application No. 16/584,737 is a continuation-in-part of application No. PCT/US18/49400, filed on Sep. 4, 2018.
Application No. 17/694,366 is a continuation-in-part of application No. 16/584,737, filed on Sep. 26, 2019.
Application No. 16/584,737 is a continuation-in-part of application No. PCT/US18/49400, filed on Sep. 4, 2018.
Application No. 17/694,366 is a continuation-in-part of application No. 16/584,737, filed on Sep. 26, 2019.
Application No. 17/676,711 is a continuation-in-part of application No. 16/584,737, filed on Sep. 26, 2019.
Application No. 16/584,737 is a continuation-in-part of application No. 16/810,204, filed on Mar. 5, 2020.
Application No. 16/810,204 is a continuation-in-part of application No. PCT/US18/49400, filed on Sep. 4, 2018.
Application No. 17/694,366 is a continuation-in-part of application No. 16/810,204, filed on Mar. 5, 2020.
Application No. 17/676,711 is a continuation-in-part of application No. 16/810,204, filed on Mar. 5, 2020.
(60) Provisional application No. 62/958,517, filed on Jan. 8, 2020.
(60) Provisional application No. 62/958,634, filed on Jan. 8, 2020.
(60) Provisional application No. 62/958,554, filed on Jan. 8, 2020.
(60) Provisional application No. 62/772,741, filed on Nov. 29, 2018.
(60) Provisional application No. 62/889,461, filed on Aug. 20, 2019.
(60) Provisional application No. 63/126,855, filed on Dec. 17, 2020.
(60) Provisional application No. 63/126,855, filed on Dec. 17, 2020.
(60) Provisional application No. 62/944,952, filed on Dec. 6, 2019.
(60) Provisional application No. 62/738,573, filed on Sep. 28, 2018.

(30) Foreign Application Priority Data
Sep. 7, 2017 (GB) 1714392.6

(51) Int. Cl. A61N 5/10; A61F 9/007

(52) U.S. Cl. CPC A61N 5/1017; A61F 9/00781; A61F 9/0017

(57) ABSTRACT

Methods and systems for applying beta radiation to a treatment area, such as a target area of a bleb, in association with and/or in combination with glaucoma surgery. The methods and systems herein may help achieve and/or maintain a healthy intraocular pressure, maintain functioning blebs and/or drainage holes arising from glaucoma drainage procedures or surgeries, help avoid scar formation or wound reversion, inhibit or reduce fibrogenesis and/or inflammation in the blebs or surrounding areas, etc.

### CROSS-REFERENCES TO RELATED APPLICATIONS

This application is a continuation-in-part of and claims priority to PCT Application No. PCT/US2021/012694 filed on Jan. 8, 2021, which claims priority to U.S. Provisional Application No. 62/958,517 filed Jan. 8, 2020 and U.S. Provisional Application No. 62/958,634 filed Jan. 8, 2020, the specification(s) of which is/are incorporated herein in their entirety by reference.

This application is a continuation-in-part of and claims priority to PCT Application No. PCT/US2021/012744 filed on Jan. 8, 2021, which claims priority to U.S. Provisional Application No. 62/958,554 filed on Jan. 8, 2020, the specification(s) of which is/are incorporated herein in their entirety by reference.

This application is a continuation-in-part of and claims priority to U.S. patent application Ser. No. 17/694,366 filed on Mar. 14, 2022, which is a continuation-in-part of and claims priority to U.S. patent application Ser. No. 16/698,676 filed on Nov. 27, 2019, now U.S. Pat. No. 11,273,325, which claims priority to U.S. Provisional Application No. 62/772,741 filed on Nov. 29, 2018, the specification(s) of which is/are incorporated herein in their entirety by reference.

U.S. Ser. No. 17/694,366 is also a continuation-in-part of and claims priority to PCT/US2021/012694. U.S. Ser. No. 17/694,366 is also a continuation-in-part of and claims priority to PCT/US2021/012744.

This application is a continuation-in-part of and claims priority to U.S. patent application Ser. No. 17/676,711 filed on Feb. 21, 2022, which is a continuation-in-part of and claims priority to PCT Application No. PCT/US2020/047235 filed on Aug. 20, 2020, which claims priority to U.S. Provisional Application No. 62/889,461 filed on Aug. 20, 2019, the specification(s) of which is/are incorporated herein in their entirety by reference.

U.S. Ser. No. 17/676,711 is also a continuation-in-part of and claims priority to PCT/US2021/012694. U.S. Ser. No. 17/676,711 is also a continuation-in-part of and claims priority to PCT/US2021/012744. U.S. Ser. No. 17/676,711 is also a continuation-in-part of and claims priority to U.S. Ser. No. 16/698,676. U.S. Ser. No. 17/694,366 is also a continuation-in-part of and claims priority to U.S. Ser. No. 17/676,711.

This application is a continuation-in-part of and claims priority to PCT Application No. PCT/US2021/064141 filed on Dec. 17, 2021, which also claims priority to PCT/US2021/012744, PCT/US2021/012694, and U.S. Provisional Application No. 63/126,855 filed on Dec. 17, 2020, the specification(s) of which is/are incorporated herein in their entirety by reference.

U.S. Ser. No. 17/694,366 is also a continuation-in-part of and claims priority to PCT/US2021/064141. U.S. Ser. No. 17/676,711 is also a continuation-in-part of and claims priority to PCT/US2021/064141.

This application is a continuation-in-part of and claims priority to PCT Application No. PCT/US2021/064190 filed on Dec. 17, 2021, which also claims priority to PCT/US2021/012744, PCT/US2021/012694, and U.S. Provisional Application No. 63/126,855 filed on Dec. 17, 2020, the specification(s) of which is/are incorporated herein in their entirety by reference.

U.S. Ser. No. 17/694,366 is also a continuation-in-part of and claims priority to PCT/US2021/064190. U.S. Ser. No. 17/676,711 is also a continuation-in-part of and claims priority to PCT/US2021/064190.

This application is a continuation-in-part of and claims priority to U.S. Ser. No. 17/782,940 filed Jun. 6, 2022, which is a 371 application of PCT Application No. PCT/US2020/063435 filed on Dec. 4, 2020, which claims priority to U.S. Provisional Application No. 62/944,952 filed on Dec. 6, 2019, the specification(s) of which is/are incorporated herein in their entirety by reference.

U.S. Ser. No. 17/694,366 is also a continuation-in-part of and claims priority to PCT/US2020/063435. U.S. Ser. No. 17/676,711 is also a continuation-in-part of and claims priority to PCT/US2020/063435.

This application is a continuation-in-part of and claims priority to U.S. patent application Ser. No. 16/584,737 filed Sep. 26, 2019, which claims priority to U.S. Provisional Application No. 62/738,573 filed on Sep. 28, 2018 and is also a continuation-in-part and claims priority to PCT Application No. PCT/US2018/049400 filed on Sep. 4, 2018, which claims priority to GB Application No. 1714392.6 filed Sep. 7, 2017, the specification(s) of which is/are incorporated herein in their entirety by reference.

U.S. Ser. No. 17/694,366 is also a continuation-in-part of and claims priority to U.S. Ser. No. 16/584,737. U.S. Ser. No. 17/676,711 is also a continuation-in-part of and claims priority to U.S. Ser. No. 16/584,737.

This application is a continuation-in-part of and claims priority to U.S. patent application Ser. No. 16/810,204 filed Mar. 5, 2020, which is a continuation-in-part and claims priority to PCT Application No. PCT/US2018/049400 filed on Sep. 4, 2018, which also claims priority to GB Application No. 1714392.6, the specification(s) of which is/are incorporated herein in their entirety by reference.

U.S. Ser. No. 17/694,366 is also a continuation-in-part of and claims priority to U.S. Ser. No. 16/810,204. U.S. Ser. No. 17/676,711 is also a continuation-in-part of and claims priority to U.S. Ser. No. 16/810,204.

### FIELD OF THE INVENTION

The present invention relates to methods, systems, and compositions for treating glaucoma treatment-associated drainage blebs and/or channels, such as those associated with foreign bodies or other glaucoma procedures, for maintaining functioning drainage blebs and/or channels, for lowering intraocular pressure, for achieving a healthy intraocular pressure, etc., with the use of beta radiation.

### BACKGROUND OF THE INVENTION

### Glaucoma

Glaucoma is the leading cause of irreversible blindness and represents a family of diseases with a characteristic optic neuropathy. Therapy for this group of diseases is principally focused at reducing the intraocular pressure (IOP) of the fluid inside the eye (aqueous humor), thus averting ongoing damage to the optic nerve.

Glaucoma is managed by attempting to lower the intraocular pressure (IOP). In the USA, Europe, and some other industrialized countries, the first line therapy is typically medication delivered by eye drops. Such medications include beta-blockers, prostaglandins, alpha-adrenergic agonists, and carbonic anhydrase inhibitors. For patients who fail medication and in other parts of the world where there are economic and distribution barriers to the practicality of daily medication and frequent follow up, the treatment regime is primarily surgical interventions.

One way to prevent vision loss from glaucoma is to lower intraocular pressure with drainage surgery that shunts fluid out of the eye through a channel created during a trabeculectomy procedure, by implanting a flow-controlled drainage device during Minimally Invasive Glaucoma Surgery (MIGS), or by the use of other surgical procedures such as Minimally Invasive Micro Sclerostomy (MIMS), trabeculectomy, or other devices. These systems and procedures allow drainage of the aqueous humor from within the eye to a small reservoir (termed a “bleb”) under the conjunctiva, from where the aqueous humor is later reabsorbed.

With current glaucoma treatments (e.g., MIMS, MIGS, trabeculectomy, etc.), scar tissue often compromises the bleb or other surrounding structures (e.g., drainage channels associated with MIMS), ultimately impeding or blocking the flow of excess fluid. Despite compelling therapeutic advantages over nonsurgical treatments, drainage surgery and devices are clinically limited by postoperative scarring.

Attempts to address this include the application of antimetabolites such as mitomycin C (MMC) and 5-fluorouracil (5FU). These antimetabolites are used in liquid form and are delivered either by injection or by placing microsurgical sponges soaked in the drug directly onto the operative site underneath the conjunctiva. One of the problems associated with antimetabolites (e.g., MMC and 5FU) is that they do not preserve blebs well. By some reports, the failure rate by three years approaches 50%.

### SUMMARY OF THE INVENTION

The present invention features methods and systems for applying radiation to a treatment area, such as a target area of a bleb, in combination with glaucoma. The methods and systems herein may be used to apply beta radiation to a target area in the eye to help maintain functioning blebs and/or drainage holes arising from glaucoma drainage procedures or surgeries, to help avoid scar formation or wound reversion, to inhibit or reduce fibrogenesis and/or inflammation in the blebs or surrounding areas, etc. The present invention is not limited to the applications disclosed herein.

The methods feature applying a therapeutic dose of beta radiation to the target site (e.g., drainage bleb, foreign body, drainage channel, and/or other appropriate site) before and/or during and/or after the time of glaucoma surgery (e.g., implantation of a drainage device, e.g., MIGS implantation).

The methods herein feature the use of an applicator for applying the therapeutic dose of beta radiation to the target site, wherein the applicator system has a distal end that contacts the target site. The methods herein feature pressing upon the applicator such that at least a portion of the conjunctiva edema fluid present at the target site is pushed away, causing a blanching effect, e.g., whitening of the area. FIG. 3, FIG. 4, FIG. 5, FIG. 6, and FIG. 7 show the blanching effect and the progression of blanching as more pressure is progressively applied.

All or a portion of the outer surface of the applicator system may be in contact with the eye. For example, in certain embodiments, at least 25% of the surface area of the outer surface of the applicator system is in contact with the eye, e.g., the target area. In certain embodiments, at least 50% of the surface area of the outer surface of the applicator system is in contact with the eye, e.g., the target area. In certain embodiments, at least 75% of the surface area of the outer surface of the applicator system is in contact with the eye, e.g., the target area. In certain embodiments, at least 90% of the surface area of the outer surface of the applicator system is in contact with the eye, e.g., the target area. In certain embodiments, at least 95% of the surface area of the outer surface of the applicator system is in contact with the eye, e.g., the target area.

The present invention also features a radioisotope, a composition, a system, etc. that emits beta radiation for use in the methods herein, e.g., methods of treating glaucoma, methods of lowering intraocular pressure, methods of maintaining functioning drainage blebs and/or channels, methods of preventing or reducing scar formation in a drainage bleb or drainage channel, etc.

As used herein, the term “treatment area” or “target area” may refer to the tissue that is desired or expected to be treated with beta radiation. The treatment area or target area may be defined as, but is not limited to, a particular plane of a certain size and a particular depth within an area of tissue being exposed to the beta radiation.

The methods and systems herein help provide a therapeutic dose of beta radiation, e.g., an optimized dose distribution across the target area or treatment area. Without wishing to limit the present invention to any theory or mechanism, as used herein, the terms “uniform dose” or “optimized dose distribution” may refer to a dose across a particular plane of a certain size at a particular depth on or within the target area or treatment area that is substantially uniform and therapeutic in dose. For example, the dose across the particular plane on or within the target may vary by no more than a certain percentage of the average or maximum dose. FIG. 1 the present invention shows a relatively flat and consistent dose across a large portion of the target area. FIG. 2 illustrates a non-limiting example of a plane of a target area, e.g., the distal end of an applicator system (with the radiation source therein, e.g., radionuclide brachytherapy source (RBS)), is in contact with the eye, and the radiation is emitted to a particular target plane within the target/treatment area. The target plane is a particular distance from the RBS and a particular distance from the top of the target area.

The size and dimensions (and depth) of the target and target plane may vary. In some embodiments, the diameter of the target area is 6 mm. In some embodiments, the diameter of the target area is 7 mm. In some embodiments, the diameter of the target area is 8 mm. In some embodiments, the diameter of the target area is 9 mm. In some embodiments, the diameter of the target area is 10 mm. In some embodiments, the diameter of the target area is 11 mm. In some embodiments, the depth of the target area, e.g., the depth of a plane of the target area, is 0 mm (e.g., in contact with a brachytherapy deliver system, e.g., a radionuclide brachytherapy system). In some embodiments, the depth of the target area, e.g., the depth of a plane of the target area, is 0.1 mm. In some embodiments, the depth of the target area, e.g., the depth of a plane of the target area, is 0.2 mm. In some embodiments, the depth of the target area, e.g., the depth of a plane of the target area, is 0.3 mm. In some embodiments, the depth of the target area, e.g., the depth of a plane of the target area, is 0.4 mm. In some embodiments, the depth of the target area, e.g., the depth of a plane of the target area, is 0.5 mm. In some embodiments, the depth of the target area, e.g., the depth of a plane of the target area, is 0.6 mm. In some embodiments, the depth of the target area is from 0 to 0.4 mm.

Alternatively, “optimized dose distribution” may also mean that the dose distribution is varied across the lesion in a specific pattern with the intention to best affect the therapeutic outcome. In one example, the dose distribution across the diameter/plane at the treatment depth varies such that the areas at the edges of the bleb receive a higher dose relative to the center. In one example, the dose distribution across the diameter/plane at the treatment depth varies such that the area at the MIGS device outflow orifice receives a boosted dose compared to other areas. In one example, the dose distribution across the diameter/plane at the treatment depth varies such that the edges of the bleb and also the area at the MIGS device outflow orifice both receive a boosted dose. In one example, the dose is attenuated over a specified area. In one example, the dose is attenuated over the cornea.

Beta radiation attenuates quickly with depth. In some embodiments, the term “optimized dose distribution” includes an appropriate dose through the depth of the target tissue. The clinical dosage depth may be determined by the thickness of the conjunctiva and associated tenon's capsule of a functional bleb. As a non-limiting example, for MIGS surgery, the focus area may be approximately 3 mm above the superior limbus. Howlet et al., found the mean thickness of the conjunctival and Tenon's layer to be 393±67 microns ranging from 194 to 573 microns using optical coherence tomography (OCT) in glaucoma patients (Howlet J et al., Journal of Current Glaucoma Practice 2014, 8(s):63-66). In an earlier study, Zhang et al. found conjunctival thickness to be 238±51 microns in healthy individuals using OCT analysis and concluded OCT accurately measures the cross-sectional structures of conjunctival tissue with high resolution (Zhang et al., Investigative Ophthalmology & Visual Science 2011, 52(10):7787-7791). Based on the Howlet study, the target tissue thickness may range from 150 to 700 microns, or from 10 to 700 microns, etc. In one example, the dose distribution from the surface through the depth of the target tissue allows for a therapeutic dose within the tissue to the limits of the rapidly attenuating beta rays.

Referring to any of the embodiments herein, in certain embodiments, the therapeutic dose of beta radiation is from 250-1000 cGy. In certain embodiments, the therapeutic dose of beta radiation is from 450-3200 cGy. In certain embodiments, the therapeutic dose of beta radiation is from 250-1100 cGy. In certain embodiments, the therapeutic dose of beta radiation is from 500-3200 cGy.

The therapeutic dose of beta radiation may be applied in one dose. In certain embodiments, the therapeutic dose of beta radiation is fractionated and applied via multiple doses. As a non-limiting example, the therapeutic dose of beta radiation may be administered weekly for 3 weeks, e.g., 800 cGy per week for 3 weeks.

Referring to any of the embodiments herein, the method may comprise performing a glaucoma drainage surgery on an eye of a patient that forms a bleb in a subconjunctival space or between the conjunctiva and Tenon's capsule and the glaucoma drainage surgery allows aqueous humor to drain into the bleb (e.g., MIGS, MIMS, trabeculectomy, etc.). In some embodiments, the methods herein comprises performing a glaucoma drainage surgery on an eye of a patient wherein an implant (e.g., MIGS implant) is implanted trans-sclerally to form a bleb in a subconjunctival space or between the conjunctiva and Tenon's capsule, the glaucoma drainage surgery allows aqueous humor to drain into the bleb. In some embodiments, the glaucoma surgery is Minimally Invasive Glaucoma Surgery (MIGS). In some embodiments, the glaucoma surgery is Minimally Invasive Micro Sclerostomy (MIMS). In some embodiments, the glaucoma surgery is trabeculectomy.

Note in some embodiments, the glaucoma drainage surgery has previously been performed.

The method comprises applying a therapeutic dose of beta radiation (e.g., from a radioisotope, system, composition, etc.) to a target area of the eye. In certain embodiments, the target area is associated with the bleb, a glaucoma drainage implant, or a drainage channel, or a combination thereof, etc. In some embodiments, the target area is associated with the bleb, the implant, or both the bleb and implant.

In some embodiments, the radioisotope (or composition or system) comprises Strontium-90 (Sr-90), Phosphorus-32 (P-32), Ruthenium 106 (Ru-106), Yttrium 90 (Y-90), or a combination thereof. In some embodiments, the therapeutic dose is from 500-1000 cGy.

Referring to the methods disclosed herein, in certain embodiments, the therapeutic dose of beta radiation helps maintain a functioning drainage bleb. In certain embodiments, the therapeutic dose of beta radiation helps maintain a functioning drainage bleb. In certain embodiments, the therapeutic dose of beta radiation causes cell cycle arrest in fibroblasts (e.g., on the Tenon's capsule) to inhibit or reduce the fibrotic process and conjunctival inflammation. In certain embodiments, the therapeutic dose of beta radiation helps reduce conjunctival inflammation. In certain embodiments, the therapeutic dose of beta radiation helps reduce fibrotic processes and conjunctival inflammation. In certain embodiments, the therapeutic dose of beta radiation helps achieve a healthy IOP. In certain embodiments, the therapeutic dose of beta radiation helps reduce intraocular pressure (IOP). In certain embodiments, the therapeutic dose of beta radiation helps inhibit or reduce fibrogenesis and inflammation in the bleb, around the drainage implant, or around the drainage channel. In some embodiments, the therapeutic dose of beta radiation helps reduce conjunctival inflammation.

Referring to any of the embodiments herein, in some embodiments, the method further comprises administering a drug to the target area. In some embodiments, the drug an anti-metabolite, e.g., mitomycin C, 5-fluorouracil. In some embodiments, the drug is an anti-VEGF composition.

Referring to any of the embodiments herein, in some embodiments, beta radiation is applied to the target after performing the glaucoma drainage surgery. In some embodiments, beta radiation is applied to the target before performing the glaucoma drainage surgery. In some embodiments, beta radiation is applied to the target while performing the glaucoma drainage surgery. In some embodiments, beta radiation is applied to the target before and after performing the glaucoma drainage surgery. In certain embodiments, beta radiation is applied before and during surgery, before and after surgery, during and after surgery, etc.

Referring to any of the embodiments herein, in some embodiments, IOP is reduced to 12 mmHg or less. In some embodiments, IOP is reduced to 10 mmHg or less. In some embodiments, IOP is reduced to from 5 to 10 mmHg. In some embodiments, IOP is reduced to from 5 to 12 mmHg. In some embodiments, IOP is reduced to from 8 to 10 mmHg. In some embodiments, IOP is reduced to from 8 to 12 mmHg.

Referring to any of the embodiments herein, the method may be effective for reducing IOP by a certain amount for a certain length of time after treatment. In some embodiments, the method is effective for reducing IOP by 20% or more 6 months after treatment. In some embodiments, the method is effective for reducing IOP by 30% or more 6 months after treatment. In some embodiments, the method is effective for reducing IOP by 40% or more 6 months after treatment. In some embodiments, the method is effective for reducing IOP by 50% or more 6 months after treatment. In some embodiments, the method is effective for reducing IOP by 20% or more 12 months after treatment. In some embodiments, the method is effective for reducing IOP by 30% or more 12 months after treatment. In some embodiments, the method is effective for reducing IOP by 40% or more 12 months after treatment. In some embodiments, the method is effective for reducing IOP by 50% or more 12 months after treatment. In some embodiments, the method is effective for reducing IOP by 20% or more 24 months after treatment. In some embodiments, the method is effective for reducing IOP by 30% or more 24 months after treatment. In some embodiments, the method is effective for reducing IOP by 40% or more 24 months after treatment. In some embodiments, the method is effective for reducing IOP by 50% or more 24 months after treatment. In some embodiments, the method is effective for reducing IOP by 20% or more 36 months after treatment. In some embodiments, the method is effective for reducing IOP by 30% or more 36 months after treatment. In some embodiments, the method is effective for reducing IOP by 40% or more 36 months after treatment. In some embodiments, the method is effective for reducing IOP by 50% or more 36 months after treatment.

Referring to any of the embodiments herein, in some embodiments, the method is effective for reduction of IOP and subsequent stabilization of IOP, e.g., IOP is stabilized for a certain length of time. In some embodiments, stabilization of IOP is wherein the IOP does not increase by more than 10% at 3 months after treatment. In some embodiments, stabilization of IOP is wherein the IOP does not increase by more than 10% at 6 months after treatment. In some embodiments, stabilization of IOP is wherein the IOP does not increase by more than 10% at 12 months after treatment. In some embodiments, stabilization of IOP is wherein the IOP does not increase by more than 10% at 24 months after treatment. In some embodiments, stabilization of IOP is wherein the IOP does not increase by more than 10% at 36 months after treatment. In some embodiments, stabilization of IOP is wherein the IOP does not increase by more than 20% at 3 months after treatment. In some embodiments, stabilization of IOP is wherein the IOP does not increase by more than 20% at 6 months after treatment. In some embodiments, stabilization of IOP is wherein the IOP does not increase by more than 20% at 12 months after treatment. In some embodiments, stabilization of IOP is wherein the IOP does not increase by more than 20% at 24 months after treatment. In some embodiments, stabilization of IOP is wherein the IOP does not increase by more than 20% at 36 months after treatment. In some embodiments, stabilization of IOP is wherein the IOP does not increase by more than 25% at 24 months after treatment. In some embodiments, stabilization of IOP is wherein the IOP does not increase by more than 25% at 36 months after treatment.

In some embodiments, inhibiting or reducing fibrogenesis and inflammation in the bleb is measured according to a predetermined bleb grading scale. The predetermined bleb grading scale is the Moorfields bleb grading scale (MBGS) and/or the Indiana Bleb Appearance Grading Scale (IBAGS).

In some embodiments, the beta radiation is applied to the target using an applicator.

In some embodiments, the target is at least a portion of a bleb. In some embodiments, the target comprises an entire bleb. In some embodiments, the target area surrounds an end of a Minimally Invasive Glaucoma Surgery (MIGS) implant. In some embodiments, the target comprises at least a portion of the bleb above a drainage channel. In some embodiments, the target further comprises at least a portion of the bleb above a drainage channel and at least a portion of a perimeter of the bleb. In some embodiments, the target further comprises at least a portion of the bleb above a drainage channel, at least a portion of a perimeter of the bleb, and at least a portion of the bleb between the perimeter and the portion above the drainage channel. In some embodiments, the target comprises a portion of a bleb. In some embodiments, the target area comprises an end of a Minimally Invasive Glaucoma Surgery (MIGS) implant.

Referring to any of the embodiments herein, in some embodiments, the method is effective for preventing further loss of vision for a certain time period. Loss of vision may be determined using techniques, measurements, and scales well known to one of ordinary skill in the art. In some embodiments, the method prevents further loss of vision for at least 2 months after treatment. In some embodiments, the method prevents further loss of vision for at least 3 months after treatment. In some embodiments, the method prevents further loss of vision for at least 4 months after treatment. In some embodiments, the method prevents further loss of vision for at least 5 months after treatment. In some embodiments, the method prevents further loss of vision for at least 6 months after treatment. In some embodiments, the method prevents further loss of vision for at least 7 months after treatment. In some embodiments, the method prevents further loss of vision for at least 8 months after treatment. In some embodiments, the method prevents further loss of vision for at least 9 months after treatment. In some embodiments, the method prevents further loss of vision for at least 12 months after treatment. In some embodiments, the method prevents further loss of vision for at least 18 months after treatment. In some embodiments, the method prevents further loss of vision for at least 24 months after treatment.

The present invention also features a radionuclide brachytherapy source (RBS) system that emits beta radiation for use in a method of treating glaucoma (e.g., for helping to lower IOP). In some embodiments, the method comprises performing a glaucoma drainage surgery on an eye of a patient to form a bleb in a subconjunctival space or between the conjunctiva and Tenon's capsule, and to allow aqueous humor to drain into the bleb; and applying a therapeutic dose of beta radiation from the RBS system to a target area associated with the bleb, a drainage channel, a drainage implant, or a combination thereof. In some embodiments, the glaucoma drainage surgery is MIGS, MIMS, or trabeculectomy.

The present invention also features a method of reducing intraocular pressure (IOP) in an eye being treated or having been treated with glaucoma drainage surgery to form a bleb in a subconjunctival space or between the conjunctiva and Tenon's capsule and to allow aqueous humor to drain into the drainage bleb. In some embodiments, the method comprises applying a therapeutic dose of beta radiation from a radionuclide brachytherapy (RBS) system to a target area associated with the bleb, a drainage channel, a drainage implant, or a combination thereof. In some embodiments, the glaucoma drainage surgery is MIGS, MIMS, or trabeculectomy.

The present invention also features methods using applicator systems described herein, e.g., with a radiation source (e.g., RBS, radioisotope, etc.) disposed at its distal end. In certain embodiments, the outer surface of the distal end (e.g., the surface that is to be in contact with the eye) may be flat or substantially flat. In some embodiments, the outer surface of the distal end (e.g., the surface that is to be in contact with the eye) has curvature (e.g., convex, concave). In some embodiments, a portion of the outer surface has curvature and a portion of the outer surface is flat. The outer surface is pressed against the surface of the eye over the target area, e.g., the bleb. The fluid associated with conjunctiva edema (or a portion thereof) is evacuated, creating a uniform distance between the outer surface of the applicator and the bottom surface of the bleb. The fluid need not be a collection or puddle of free fluid, but in certain embodiments, the fluid could be interstitial edema or fluid that is within the tissue of the conjunctiva.

Without wishing to limit the present invention to any theory or mechanism, one benefit of removing (or reducing) the edema fluid is that the conjunctiva tissue to be irradiated is then more uniform in thickness, and this helps allow for the use of standard radiation dosimetry. (Otherwise, in edematous tissue or tissue of unknown thickness that varies from patient to patient, there would be little way to know how long to apply the brachytherapy applicator to ensure the intended minimum dose is delivered to all the target tissue. Because beta is attenuated over very short distances of a fraction of a millimeter, more edematous tissue would be thicker and the beta radiation would thus be attenuated and may not reach the more distal tissue with a strong enough dose.) Note that while Castroviejo masks had a flat outer surface, they were not used from edge to edge. The masks limited most of the radiation emission to a very small area with respect to the total surface area of the outer surface of the mask. In the present invention, the outer surface refers to the area at the end of the distal end of the applicator through which the therapeutic dose of radiation is emitted.

The present invention also features a brachytherapy system comprising an applicator, the applicator having a handle and a distal end with an outer surface; and a radioisotope that emits beta radiation disposed in the distal end, the beta radiation is emitted through the outer surface of the distal end; wherein the outer surface is flat.

The present invention also features a brachytherapy system comprising an applicator, the applicator having a handle and a distal end with an outer surface; and a radioisotope that emits beta radiation disposed in the distal end, the beta radiation is emitted through the outer surface of the distal end; wherein the outer surface has a convex curvature.

The present invention also features a brachytherapy system comprising an applicator, the applicator having a handle and a distal end with an outer surface; and a radioisotope that emits beta radiation disposed in the distal end, the beta radiation is emitted through the outer surface of the distal end; wherein the outer surface has a concave curvature.

Described herein is a method of inhibiting or reducing fibrogenesis and inflammation in a bleb of an eye being treated for glaucoma, the bleb being in the subconjunctival space of the eye or in a space between the conjunctiva and Tenon's capsule, said method comprising applying a therapeutic dose of beta radiation from a radioisotope to a target area of the eye using an applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away, e.g., a blanching effect; wherein the therapeutic dose of beta radiation causes cell cycle arrest in fibroblasts on the Tenon's capsule to inhibit or reduce the fibrotic process and inflammation that leads to bleb failure.

Described herein is a method of maintaining a functioning drainage bleb in the eye of a patient being treated for glaucoma, the method comprising applying a therapeutic dose of beta radiation from a radioisotope to a target area of the eye using an applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away, e.g., a blanching effect; wherein the therapeutic dose of beta radiation reduces or inhibits a fibrotic process and inflammation that causes bleb failure, and wherein the method is effective to maintain the drainage function of the bleb.

Described herein is a method of treating glaucoma, the method comprising applying a therapeutic dose of beta radiation from a radioisotope to a target area of the eye using an applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away, e.g., a blanching effect; wherein the method is effective for reducing an Intraocular Pressure (IOP) of the eye.

Described herein is a method of reducing intraocular pressure (IOP) in an eye, said method comprising applying a therapeutic dose of beta radiation from a radioisotope to a target area of the eye using an applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away, e.g., a blanching effect; wherein the therapeutic dose of beta radiation is effective for reducing an Intraocular Pressure (IOP) of the eye.

Described herein is a method of reducing inflammation in an eye having a foreign body therein, said method comprising applying a therapeutic dose of beta radiation from a radioisotope to a target area of the eye using an applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away, e.g., a blanching effect; wherein the method is effective for reducing inflammation caused by the presence of the foreign body.

Referring to the embodiments herein, the applicator system described as having a distal end with an outer surface may refer to a single-piece or multi-piece system. For example, in some embodiments, the applicator system exists as an applicator with a radioisotope integrated into a distal end. In some embodiments, the distal end comprises two or more pieces, e.g., a cap may engage a radionuclide brachytherapy source and attach to the remainder of the applicator system. Thus, the distal end may refer to an attachable cap. In some embodiments, the applicator system features a cap that is disposable.

The present invention describes a beta radiation source for irradiating a target area of a human eye and a brachytherapy system for use in reducing scar formation in a draining bleb in a human eye being treated for glaucoma, wherein the drainage bleb is in a subconjunctival space of the eye or a space between the conjunctiva and Tenon's capsule by a transscleral implant. The present invention also describes a beta radiation source for irradiating a target area of a human eye and a brachytherapy system, and a transscleral implant for forming a drainage bleb in a subconjunctival space of the eye or a space between the conjunctiva and Tenon's capsule, for simultaneous, separate or sequential use in reducing scar formation in a draining bleb in a human eye being treated for glaucoma. The applicator system may comprise a handle and a distal end with the beta radiation source embedded or engaged therein; the distal end has an outer surface. In certain embodiments, the outer surface of the distal end of the applicator system is flat. In certain embodiments, the outer surface of the distal end of the applicator system has curvature. In certain embodiments, the curvature is a convex curvature. In certain embodiments, the curvature is a concave curvature. In certain embodiments, the outer surface has a portion that has curvature and a portion that is flat. In certain embodiments, the outer surface has a radius of curvature from 120 mm to flat. In certain embodiments, the outer surface has a radius of curvature from 120 mm to 1,000 mm. In certain embodiments, the outer surface of the distal end is 12 mm in diameter. In certain embodiments, the outer surface of the distal end is from 8 to 10 mm in diameter. In certain embodiments, the outer surface of the distal end is from 10 to 12 mm in diameter. In certain embodiments, the outer surface of the distal end is from 7 to 14 mm in diameter. In certain embodiments, the beta radiation source comprises Strontium-90 (Sr-90), Phosphorus-32 (P-32), Ruthenium 106 (Ru-106), Yttrium 90 (Y-90), or a combination thereof. In certain embodiments, the system further comprises a drug. In certain embodiments, the drug is an antimetabolite. In certain embodiments, the antimetabolite is mitomycin C. In certain embodiments, the antimetabolite 5 fluorouracil. In certain embodiments, the implant is a Minimally Invasive Glaucoma Surgery (MIGS) implant.

The present invention also describes a system for use in a method of treating glaucoma in an eye having been treated with glaucoma drainage surgery wherein an implant was implanted trans-scierally to form a bleb in a subconjunctival space or between a conjunctiva and Tenon's capsule and aqueous humor drains into the drainage bleb, said system comprising: a beta radiation source; and a brachytherapy applicator system. In certain embodiments, the applicator system comprises a handle and a distal end with the beta radiation source embedded or engaged therein, the distal end has an outer surface. In certain embodiments, the outer surface of the distal end of the applicator system is flat. In certain embodiments, the outer surface of the distal end of the applicator system has curvature. In certain embodiments, the curvature is a convex curvature. In certain embodiments, the curvature is a concave curvature.

In certain embodiments, the outer surface has a portion that has curvature and a portion that is flat. In certain embodiments, the outer surface has a radius of curvature from 120 mm to flat. In certain embodiments, the outer surface has a radius of curvature from 120 mm to 1,000 mm. In certain embodiments, the outer surface of the distal end is 12 mm in diameter. In certain embodiments, the outer surface of the distal end is from 8 to 10 mm in diameter. In certain embodiments, the outer surface of the distal end is from 10 to 12 mm in diameter. In certain embodiments, the outer surface of the distal end is from 7 to 14 mm in diameter. In certain embodiments, the beta radiation source comprises Strontium-90 (Sr-90), Phosphorus-32 (P-32), Ruthenium 106 (Ru-106), Yttrium 90 (Y-90), or a combination thereof.

The present invention also describes a system for use in a method of treating glaucoma, said system comprising a beta radiation source and a brachytherapy applicator system, the method comprising: performing a glaucoma drainage surgery on an eye of a patient wherein an implant is implanted trans-sclerally to form a bleb in a subconjunctival space or between the conjunctiva and Tenon's capsule, the glaucoma drainage surgery allows aqueous humor to drain into the bleb; and applying a therapeutic dose of beta radiation from the beta radiation source to a target area of the eye using the applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away; wherein the method is effective for lowering intraocular pressure (IOP). The present invention also describes a system for use in a method of treating glaucoma, said system comprising a beta radiation source and a brachytherapy applicator system, the method comprising: applying a therapeutic dose of beta radiation from the beta radiation source to a target area of the eye using the applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away; wherein the method is effective for lowering intraocular pressure (IOP). In certain embodiments, a Minimally Invasive Glaucoma Surgery (MIGS) implant is inserted trans-sclerally. In certain embodiments, the distance from the outer surface of the distal end of the applicator system and the bottom surface of the bleb is substantially uniform across the target area. In certain embodiments, the outer surface of the distal end of the applicator system is flat. In certain embodiments, the outer surface of the distal end of the applicator system has curvature. In certain embodiments, the curvature is a convex curvature. In certain embodiments, the curvature is a concave curvature. In certain embodiments, the outer surface has a portion that has curvature and a portion that is flat. In certain embodiments, the outer surface has a radius of curvature from 120 mm to flat. In certain embodiments, the outer surface has a radius of curvature from 120 mm to 1,000 mm. In certain embodiments, the outer surface of the distal end is 12 mm in diameter. In certain embodiments, the outer surface of the distal end is from 8 to 10 mm in diameter. In certain embodiments, the outer surface of the distal end is from 10 to 12 mm in diameter. In certain embodiments, the outer surface of the distal end is from 7 to 14 mm in diameter. In certain embodiments, at least 25% of the surface area of the outer surface of the distal end is in contact with the eye. In certain embodiments, at least 50% of the surface area of the outer surface of the distal end is in contact with the eye. In certain embodiments, at least 75% of the surface area of the outer surface of the distal end is in contact with the eye. In certain embodiments, at least 90% of the surface area of the outer surface of the distal end is in contact with the eye. In certain embodiments, the target comprises an entire bleb. In certain embodiments, the target area surrounds an end of a MIGS implant. In certain embodiments, the radioisotope comprises Strontium-90 (Sr-90), Phosphorus-32 (P-32), Ruthenium 106 (Ru-106), Yttrium 90 (Y-90), or a combination thereof. In certain embodiments, the therapeutic dose of beta radiation is from 250-1000 cGy. In certain embodiments, the therapeutic dose of beta radiation is from 450-3200 cGy. In certain embodiments, the therapeutic dose of beta radiation is from 250-1100 cGy. In certain embodiments, the therapeutic dose of beta radiation is from 500-3200 cGy. In certain embodiments, the method further comprises administering a drug to the target area. In certain embodiments, the drug is an antimetabolite. In certain embodiments, the antimetabolite is mitomycin C. In certain embodiments, the antimetabolite 5 fluorouracil. In certain embodiments, the drug is an anti-VEGF composition. In certain embodiments, the step of pressing the outer surface of the distal end of the applicator system causes blanching of tissue underneath the outer surface. In certain embodiments, the method is effective for reducing IOP to 12 mmHg or less. In certain embodiments, the method is effective for reducing IOP by 20% or more 6 months after treatment. In certain embodiments, the method is effective for reducing IOP by 20% or more 12 months after treatment. In certain embodiments, the method is effective for reducing IOP by 20% or more 24 months after treatment. In certain embodiments, the method is effective for reducing IOP and subsequent stabilization of said IOP. In certain embodiments, stabilization of IOP is wherein the IOP does not increase by more than 20% at 3 months after treatment. In certain embodiments, stabilization of IOP is wherein the IOP does not increase by more than 20% at 6 months after treatment. In certain embodiments, stabilization of IOP is wherein the IOP does not increase by more than 20% at 12 months after treatment.

As used herein, the beta radiation source may be considered a consumable agent.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a comparison of a dose profile from a legacy device (prior art) and a dose profile of a system of the present invention.

FIG. 2 shows an example of a target plane within a target area, relative to a radionuclide brachytherapy system (RBS system).

FIG. 3 shows contacting the patient's eye (over the bleb) with a syringe plunger (with a flat outer surface), which as is shown in FIG. 4, FIG. 5, FIG. 6, and FIG. 7, is used to show the ability to evacuate the edema fluid.

FIG. 4 shows the syringe plunger of FIG. 3 pushed slightly against the eye over the bleb such that some of the fluid below the syringe plunger is pushed away.

FIG. 5 shows the syringe plunger FIG. 4 pressed further; more of the fluid below the syringe plunger is pushed away.

FIG. 6 shows the syringe plunger FIG. 5 pressed further; more of the fluid below the syringe plunger is pushed away.

FIG. 7 shows the syringe plunger of FIG. 6 pressed further. At this level of pressure, all of the fluid below the syringe plunger is pushed away.

FIG. 8 shows non-limiting examples of distal ends of applicators, e.g., applicator A, applicator B, applicator C, and applicator D (not drawn to scale).

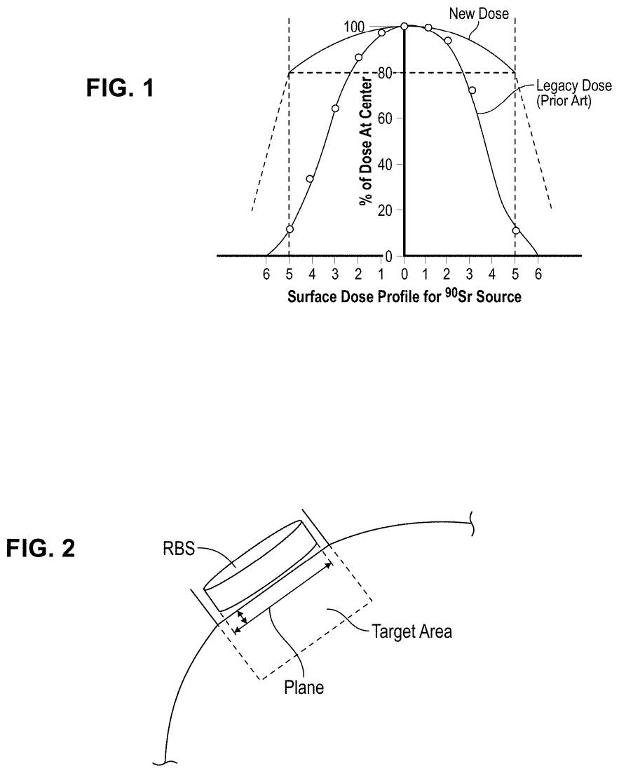

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

All embodiments disclosed herein can be combined with other embodiments unless the context clearly dictates otherwise.

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references

Dosimetry techniques include film dosimetry. In one example the RBS is applied to radiographic film, for example Gafchromic™ film. The dose at various depths can also be measured by placing an intervening material, such as Plastic Water™, of known thicknesses between the RBS and the film. A transmission densitometer in conjunction with a film optical density vs. dose chart, allows for the film opacity to be measured and then converted to the delivered dose. Other methods include Thermoluminescent methods (TLD chips). TLD chips are small plastic chips with millimeter dimensions having a crystal lattice that absorbs ionizing radiation.

Dose variation is described as that across the diameter, assuming a central point maximum dose. However, in practice it has been demonstrated that the maximum dose may be off center. Thus, a description of variation of dose across the diameter may also include the variation of dose over the area, and through the depth.

In general use in the profession of ophthalmology the term “conjunctivae” may refer to the conjunctivae in combination with the Tenon's capsule. Also, in general use in the profession of ophthalmology the term “conjunctivae” may refer to the conjunctivae alone, not including the Tenon's capsule. References herein to “conjunctivae” can include either and/or both meanings.

All publications, patent applications, patents, and other references mentioned herein are incorporated by reference in their entirety for all purposes. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of the disclosure, the following explanations of specific terms are provided:

Beam Modification: Desirable modification in the spatial distribution of radiation (e.g., within the patient) by insertion of any material in the beam path. Beam modification increases conformity allowing a higher dose delivery to the target, while sparing more normal tissue simultaneously. There are four main types of beam modification: (1) Shielding: To eliminate radiation dose to some special parts of the zone at which the beam is directed. In general use is the fabrication of low-melting-temperature alloy (Lipowitz metal or Cerroblend) shielding blocks that are custom made for the individual patient and used to shield normal tissue and critical organs. For example, during total body irradiation (TBI), customized shielding blocks are positioned in front of the lungs to reduce radiation dose. (2) Compensation: To allow normal dose distribution data to be applied to the treated zone, when the beam enters obliquely through the body, or where different types of tissues are present. (3) Wedge filtration: Where a special tilt in isodose curves is obtained. (4) Flattening: Where the spatial distribution of the natural beam is altered by reducing the central exposure rate relative to the peripheral. In general use is a beam flattening filter that reduces the central exposure rate relative to that near the edge of the beam. This technique is used for linear accelerators. The filter is designed so that the thickest part is in the center. These are often constructed of copper or brass.

Innovations such as stereotaxic radiotherapy, intensity modulated radiation therapy, and conformal radiotherapy are also applied towards the goal of sparing normal tissue and critical organs. For example, Linear Accelerators designed with Multileaf Collimators have, in many circumstances, replaced shielding bocks.

Brachytherapy (see also Radionuclide Brachytherapy Source (RBS): According to the American Association of Physicists in Medicine (AAPM), brachytherapy is “the clinical use of small encapsulated radioactive sources at a short distance from the target volume for irradiation of malignant tumors or nonmalignant lesions.” Generally, in medical practice, brachytherapy can be categorized as topical or plaque brachytherapy, intracavitary, and interstitial.

Some implementations of brachytherapy employ permanently implanted Radionuclide Brachytherapy Sources (RBSs). For example, in Low Dose Rate (LDR) brachytherapy for prostate cancer, a standard of care treatment, radioactive Iodine-125 RBSs are placed directly into the prostate where they remain indefinitely. In another implementation, High Dose Rate (HDR) brachytherapy TheraSpheres are infused into the arteries that feed liver tumors. These microspheres then embolize, lodging themselves in the liver's capillaries and bathing the malignancy in high levels of yttrium-90 radiation. In both these implementations, the total dose is given by consuming the entire radioisotope. Some other implementations of brachytherapy employ a transient placement of the RBS. For example, in after-loaded High Dose Rate (HDR) brachytherapy, very tiny plastic catheters are placed into the prostate gland, and a series of radiation treatments is given through these catheters. A computer-controlled machine pushes a single highly radioactive iridium-192 RBS into the catheters one by one for a specified dwell time at locations throughout the volume being irradiated. The catheters are then easily pulled out, and no radioactive material is left at the prostate gland. Another example of transient placement of an RBS includes prophylactic therapy for restenosis of coronary arteries after stent implantation. This is a non-malignant condition that has been successfully treated by placing a catheter into the coronary artery, then inserting an HDR radioactive source into the catheter and holding it there for a predetermined time in order to deliver a sufficient dose to the vessel wall.

Drainage Device or Drainage System: Any or a combination of the general and specific approaches for draining aqueous humor, such as the therapeutic and devices described herein, e.g., minimally invasive glaucoma surgery (MIGS) devices and surgery, Minimally Invasive Micro Sclerostomy (MIMS) devices and surgery, trabeculectomy surgery, sclerostomy, etc., that are employed to reduce intraocular pressure (IOP) by means of surgical intervention with or without a device.

Flow Controlled Stents (see also Minimally Invasive Glaucoma Surgery (MIGS)): Some MIGS-associated devices control flow of the aqueous humor. For example, the XEN® gel stent (Allergan) is a gelatin and glutaraldehyde tube, which is preloaded in a disposable injector and implanted using an ab interno approach. The surgeon inserts the injector through a clear cornea incision and tunnels through the sclera at or anterior to Schlemm's canal to deploy the distal portion of the stent within the subconjunctival space. This creates a pathway for aqueous to flow from the anterior chamber to the subconjunctival space, forming a bleb. Another flow-controlled stent is the InnFocus MicroShunt® (InnFocus, Santen). The surgeon inserts this device into the anterior chamber through an ab externo approach, creating a bleb in the subconjunctival space.

Functioning Drainage Bleb: A bleb that is effective for draining aqueous humor from the eye to reduce intraocular pressure (IOP) of the eye to an appropriate level.

Early bleb grading systems included those proposed by Kronfeld (1969), Migdal and Hitchings (1983), and Picht and Grehn (1998). Subsequent bleb grading systems identified and incorporated a graded assessment of various bleb parameters such as vascularity, height, width, microcystic changes, encystment and diffuse/demarcated zones.

There are two recently described grading systems for clinical grading of filtering surgery blebs: the Moorfields Bleb Grading System (MBGS) and the Indiana Bleb Appearance Grading Scale (IBAGS). The MBGS built upon the system used for this tele-medicine study and expanded it to include an assessment of vascularity away from the center of the bleb and a way to represent mixed-morphology blebs. In this scheme, central area (1-5), maximal area (1-5), bleb height (1-4) and subconjunctival blood (0-1) were assessed. In addition, three areas of the bleb were graded separately for vascularity, including bleb center conjunctiva, peripheral conjunctiva and non-bleb conjunctiva. Vascularity in each area was assigned a score from 1 to 5. A study found good inter-observer agreement and clinical reproducibility in the IBAGS and MBGS (Wells A P, Ashraff N N, Hall R C, et al. Comparison of two clinical bleb grading systems. Ophthalmology 2006; 113:77-83.)

The Moorfields bleb grading system was developed as the importance of bleb appearance to outcome was realized. Blebs that develop thin avascular zones are at increased risk of leakage and late hypotony as well as sight-threatening bleb related infections.

The Indiana Bleb Appearance Grading Scale is a system for classifying the morphologic slit lamp appearance of filtration blebs. The Indiana Bleb Appearance Grading Scale contains a set of photographic standards illustrating a range of filtering bleb morphology selected from the slide library of the Glaucoma Service at the Indiana University Department of Ophthalmology. These standards consist of slit lamp images for grading bleb height, extent, vascularity, and leakage with the Seidel test. For grading, the morphologic appearance of the filtration bleb is assessed relative to the standard images for the 4 parameters and scored accordingly.

For reference, a failed or failing bleb may have “restricted posterior flow with the so-called ‘ring of steel’,” e.g., a ring of scar tissue or fibrosis adhering the conjunctiva to the sclera at the periphery of the bleb that restricts the flow of aqueous humor (see Dhingra S, Khaw P T. The Moorfields Safer Surgery System. Middle East African Journal of Ophthalmology. 2009; 16(3):112-115). Other attributes of failed or failing blebs may include cystic appearance and/or changes in vascularization and/or scar tissue and/or thinning of the conjunctiva overlaying the bleb and/or a tense bleb and/or other observable or measurable changes as may be included in either the Indiana Bleb Appearance Grading Scale or Moorfields Bleb Grading System. Other functional determinates of failed or failing blebs or glaucoma surgery may include increased IOP, or IOP that has not decreased sufficiently.

Minimally Invasive Glaucoma Surgery (MIGS): MIGS is a recent innovation in the surgical treatment of glaucoma developed to minimize the complications from tubes and trabeculectomy. MIGS is a term applied to the widening range of implants, devices, and techniques that seek to lower intraocular pressure with less surgical risk than the more established procedures. In most cases, conjunctiva-involving devices require a subconjunctival bleb to receive the fluid and allow for its extraocular resorption. Flow-controlled conjunctiva-involving devices typically attempt to control flow and lower IOP to normal pressure and also minimizing hypotony (too low pressure in the eye) by applying Poiseuille's law of laminar flow to create a tube that is sufficiently long and narrow to restrict and control outflow. Some MIGS devices include Flow Controlled Stents, microshunts to Shlemm's Canal, Suprachoroidal Devices, and devices for Trabeculotomy. Examples of microshunts to Schlemm's Canal include iStent® (Glaukos®) and Hydrus™ (Ivantis). Examples of suprachoroidal devices include CyPass® (Alcon), Solx® gold shunt (Solx), and iStent Supra® (Glaukos). An example of a trabeculotomy device includes the Trabectome® (NeoMedix) electrocautery device.

Planning Treatment Volume or Planning Target Volume (PTV): An area or volume that encloses all the tissue intended for irradiation. The PTV includes the clinical target volume or clinical treatment volume (CTV).

Radioactive isotope, radionuclide, radioisotope: An element that has an unstable nucleus and emits radiation during its decay to a stable form. There may be several steps in the decay from a radioactive to a stable nucleus. There are four types of radioactive decay: alpha, beta negative, beta positive, and electron capture. Gamma rays can be emitted by the daughter nucleus in a de-excitation following the decay process. These emissions are considered ionizing radiation because they are powerful enough to liberate an electron from another atom.

Therapeutic radionuclides can occur naturally or can be artificially produced, for example by nuclear reactors or particle accelerators. Radionuclide generators are used to separate daughter isotopes from parent isotopes following natural decay.

Non-limiting examples of radioactive isotopes following one of the four decay processes are given herein: (1) Alpha decay: radium 226, americium 241; (2) Beta minus: iridium 192, cesium 137, phosphorous 32 (P-32), strontium 90 (Sr-90), yttrium 90 (Y-90), ruthenium 106, rhodium-106; (3) Beta positive: fluorine 18; (4) Electron capture: iodine 125, palladium 106. Examples of gamma emission include iridium 192 and cesium 137.

Half-life is defined as the time it takes for one-half of the atoms of a radioactive material to disintegrate. Half-lives for various radioisotopes can range from a few microseconds to billions of years.

The term activity in the radioactive-decay processes refers to the number of disintegrations per second. The units of measure for activity in a given source are the curie (Ci) and becquerel (Bq). One (1) Becquerel (Bq) is one disintegration per second.

An older unit is the Curie (Ci), wherein one (1) Ci is 3.7×10¹⁰ Bq.

As used herein, the term “beta radiation source,” “radiation source,” “source of beta radiation,” or “source of radiation” can refer to the term “radioisotope.” In any of the methods or compositions here, the radioisotope or source of beta radiation may comprise Strontium-90 (Sr-90), Phosphorus-32 (P-32), Ruthenium 106 (Ru-106), Yttrium 90 (Y-90), or a combination thereof.

Radionuclide Brachytherapy Source (RBS) (see also Brachytherapy): According to the US Federal Code of Regulations, a Radionuclide Brachytherapy Source (RBS) is “a device that consists of a radionuclide what may be enclosed in a sealed container made of gold, titanium, stainless steel, or platinum and intended for medical purposes to be placed onto a body surface or into a body cavity or tissue as a source of nuclear radiation for therapy.” Other forms of brachytherapy sources are also used in practice. For example, a commercially available conformal source is a flexible, thin film made of a polymer chemically bound to Phosphorous-32 (P-32). Another product is the TheraSphere, a radiotherapy treatment for hepatocellular carcinoma (HCC) that consists of millions of microscopic, radioactive glass microspheres (20-30 micrometers in diameter) containing Yttrium-90. Other forms of brachytherapy employ x-ray generators as sources instead of radioisotopes.

Sclerostomy: A procedure in which the surgeon makes a small opening in the sclera to reduce intraocular pressure (IOP), usually in patients with open-angle glaucoma. It is classified as a type of glaucoma filtering surgery. Minimally invasive micro sclerostomy (MIMS, Sanoculis) is a recent innovative technique that combines the mechanism of conventional trabeculectomy and simple needling. In the course of the surgery, a sclero-corneal drainage channel is created. The MIMS procedure can be performed ab externo by creating a sclero-corneal channel to drain the aqueous humor from the anterior chamber to the subconjunctival space. The channel created with MIMS is designed to obtain a controlled fluid flow. Laser sclerostomy can be performed in a less invasive manner than standard filtering surgery. Other studies have explored the use of laser energy of varying wavelengths, properties, and tissue interaction to create thermal sclerostomies. Several methods deliver laser energy by mirrored contact lenses to the internal face of the filtration angle or by fiberoptic cables for ab interno or ab externo sclerostomy formation.

Trabeculectomy: A procedure wherein a small hole is made in the sclera and is covered by a thin trap-door. Aqueous humor drains through the trap door to a bleb. As an example, in some trabeculectomy procedures, an initial pocket is created under the conjunctiva and Tenon's capsule and the wound bed is treated with mitomycin C soaked sponges using a “fornix-based” conjunctival incision at the corneoscleral junction. A partial thickness scleral flap with its base at the corneoscleral junction after cauterization of the flap area is created. Further, a window opening is created under the flap with a Kelly-punch or a Khaw Descemet Membrane Punch to remove a portion of the sclera, Schlemm's canal, and the trabecular meshwork to enter the anterior chamber. An iridectomy is done in many cases to prevent future blockage of the sclerostomy. The scleral flap is then sutured loosely back in place with several sutures. The conjunctiva is closed in a watertight fashion at the end of the procedure.

Trans-scleral Drainage Devices: Devices that shunt aqueous humor from the anterior chamber to a subconjunctival reservoir. As an example, the EX-PRESS® Glaucoma Filtration Device channels aqueous humor through a secure lumen to a half-thickness scleral flap, creating a subconjunctival filtration bleb. The device's lumen provides a standardized opening for aqueous humor flow while also providing some resistance, which appears to add further stability to the anterior chamber during surgery and the early post-op period.

Treat, Treatment, Treating: These terms refer to both therapeutic treatments, e.g., elimination of a disease, disorder, or condition, and prophylactic or preventative measures, e.g., preventing or slowing the development of a disease or condition, reducing at least one adverse effect or symptom of a disease, condition, or disorder, etc. Treatment may be “effective” if one or more symptoms or clinical markers are reduced as that term is defined herein. Alternatively, a treatment may be “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with a particular disease, disorder, or condition, as well as those likely to develop a particular disease, disorder, or condition due to genetic susceptibility or other factors.

Valves: Devices that can be used for glaucoma treatment, wherein instead of using a natural bleb, these devices use a synthetic reservoir (or plate), which is implanted under the conjunctiva to allow flow of aqueous fluid. Valve devices include the Baerveldt® implant (Pharmacia Co.), the Ahmed® glaucoma valve (New World Medical), the Krupin-Denver eye valve to disc implant (E. Benson Hood Laboratories), and the Molteno® and Molteno3® drainage devices (Molteno® Ophthalmic Ltd.).

### DETAILED DESCRIPTION OF THE INVENTION

The present invention provides methods and system for the application of a therapeutic dose of beta radiation to a target of an eye (associated with glaucoma surgery) for the purpose of, for example, maintaining functioning drainage blebs and/or channels, reducing fibrogenesis and/or inflammation, reducing conjunctival inflammation, lowering intraocular pressure, achieving a healthy intraocular pressure following glaucoma surgery, etc.

As used herein, the term “functioning drainage bleb” refers to a bleb that is effective for the draining of aqueous humor from the eye to reduce the IOP of the eye to an appropriate level. For example, a functioning drainage bleb may relate to a normal IOP. Early bleb grading systems included those proposed by Kronfeld (1969), Migdal and Hitchings (1983), and Picht and Grehn (1998). Subsequent bleb grading systems identified and incorporated a graded assessment of various bleb parameters such as vascularity, height, width, microcystic changes, encystment and diffuse/demarcated zones.

There are two recently described grading systems for clinical grading of filtering surgery blebs: the Moorfields Bleb Grading System (MBGS) and the Indiana Bleb Appearance Grading Scale (IBAGS). The MBGS built upon the system used for this tele-medicine study and expanded it to include an assessment of vascularity away from the center of the bleb and a way to represent mixed-morphology blebs. In this scheme, central area (1-5), maximal area (1-5), bleb height (1-4) and subconjunctival blood (0-1) were assessed. In addition, three areas of the bleb were graded separately for vascularity, including bleb center conjunctiva, peripheral conjunctiva and non-bleb conjunctiva. Vascularity in each area was assigned a score from 1 to 5. A study found good inter-observer agreement and clinical reproducibility in the IBAGS and MBGS (Wells A P, Ashraff N N, Hall R C, et al. Comparison of two clinical bleb grading systems. Ophthalmology 2006; 113:77-83.)

The Moorfields bleb grading system was developed as the importance of bleb appearance to outcome was realized. Blebs that develop thin avascular zones are at increased risk of leakage and late hypotony as well as sight-threatening bleb related infections.

The Indiana Bleb Appearance Grading Scale is a system for classifying the morphologic slit lamp appearance of filtration blebs. The Indiana Bleb Appearance Grading Scale contains a set of photographic standards illustrating a range of filtering bleb morphology selected from the slide library of the Glaucoma Service at the Indiana University Department of Ophthalmology. These standards consist of slit lamp images for grading bleb height, extent, vascularity, and leakage with the Seidel test. For grading, the morphologic appearance of the filtration bleb is assessed relative to the standard images for the 4 parameters and scored accordingly.

For reference, a failed or failing bleb may have “restricted posterior flow with the so-called ‘ring of steel’,” e.g., a ring of scar tissue or fibrosis adhering the conjunctiva to the sclera at the periphery of the bleb that restricts the flow of aqueous humor (see Dhingra S, Khaw P T. The Moorfields Safer Surgery System. Middle East African Journal of Ophthalmology. 2009; 16(3):112-115). Other attributes of failed or failing blebs may include cystic appearance and/or changes in vascularization and/or scar tissue and/or thinning of the conjunctiva overlaying the bleb and/or a tense bleb and/or other observable or measurable changes as may be included in either the Indiana Bleb Appearance Grading Scale or Moorfields Bleb Grading System. Other functional determinates of failed or failing blebs or glaucoma surgery may include increased IOP, or IOP that has not decreased sufficiently.

As used herein, the term “drainage device” refers to any or a combination of the general and specific approaches for draining aqueous humor, such as the therapeutics and devices described herein, including minimally invasive glaucoma surgery (MIGS) devices and surgery, that are employed to reduce Intraocular Pressure by means of a surgical intervention with a device.

The methods herein feature applying a therapeutic dose of beta radiation to the target site (e.g., drainage site or other appropriate site). The methods herein may also feature applying a drug to the eye, e.g., to the target, to an area near the target, etc. Non-limiting examples of drugs include mitomycin C, 5 fluorouracil, an anti-VEGF composition, and other appropriate compositions.

The methods may allow for achieving a healthy intraocular pressure (IOP). In some embodiments, the methods herein allow for achieving an IOP of 10 mmHg or less. In some embodiments, the methods herein allow for achieving an IOP of 10 mmHg. In some embodiments, the methods herein allow for achieving an IOP of 11 mmHg. In some embodiments, the methods herein allow for achieving an IOP of 12 mmHg. In some embodiments, the methods herein allow for achieving an IOP of 13 mmHg. In some embodiments, the methods herein allow for achieving an IOP of 14 mmHg. In some embodiments, the methods herein allow for achieving an IOP of 15 mmHg. In some embodiments, the methods herein allow for achieving an IOP from 10-12 mmHg. In some embodiments, the methods herein allow for achieving an IOP from 10-13 mmHg. In some embodiments, the methods herein allow for achieving an IOP from 10-14 mmHg. In some embodiments, the methods herein allow for achieving an IOP from 10-15 mmHg. In some embodiments, the methods herein allow for achieving an IOP from 9-12 mmHg. In some embodiments, the methods herein allow for achieving an IOP from 9-15 mmHg.

Various glaucoma drainage procedures and devices, including trabeculectomy, drainage tubes, and devices used for Minimally Invasive Glaucoma Surgery (MIGS), are described herein. For the purposes of the invention, other surgical innovations and/or devices in addition to those described above may be included in the scope of the invention and described and labeled as MIGS. For example, techniques and devices that may alternatively be described as Moderately Invasive Glaucoma Surgery or Augmented Incisional Surgery are also included in the present invention.

### Isotopes and Radioactivity

The US Nuclear Regulatory Commission (USNRC) (https://www.nrc.gov/about-nrc/radiation/health-effects/measuring-radiation.html) defines radioactivity as “the amount of ionizing radiation released by a material. Whether it emits alpha or beta particles, gamma rays, x-rays, or neutrons, a quantity of radioactive material is expressed in terms of its radioactivity (or simply its activity), which represents how many atoms in the material decay in a given time period. The units of measure for radioactivity are the curie (Ci) and becquerel (Bq).” Activity in a radioactive-decay process is defined as the number of disintegrations per second, or the number of unstable atomic nuclei that decay per second in a given sample. Activity is expressed in the International System of Units by the becquerel (abbreviated Bq), which is exactly equal to one disintegration per second. Another unit that may be used is the Curie, wherein one curie is approximately the activity of 1 gram of radium and equals (exactly) 3.7×10¹⁰ becquerel. The specific activity of radionuclides is relevant when it comes to selecting them for production for therapeutic pharmaceuticals.

By the USNRC definition, absorbed dose is defined as the amount of radiation absorbed, e.g., the amount of energy that radioactive sources deposit in materials through which they pass or the concentration of energy deposited in tissue as a result of an exposure to ionizing radiation. The absorbed dose is equal to the radiation exposure (ions or Ci/kg) of the radiation beam multiplied by the ionization energy of the medium to be ionized. Typically, the units for absorbed dose are the radiation absorbed dose (rad) and gray (Gy). Gy is a unit of ionizing radiation dose defined as the absorption of one joule of radiation energy per kilogram of matter. The rad has generally been replaced by the Gy in SI derived units. 1 Gy is equivalent to 100 rad.

Radionuclide generators are devices that produce a useful short-lived medical radionuclide (known as “daughter” products) from the radioactive transformation of a long-lived radionuclide (called a “parent”). By having a supply of parent on hand at a facility, the daughter is continually generated on site. The generator permits ready separation of the daughter radionuclide from the parent. One of the most widely used generator devices (often referred as a “cow”) is the technetium 99 generator. It allows the extraction of the metastable isotope 99mTc of technetium from a source of decaying molybdenum-99. 99Mo has a half-life of 66 hours and can be easily transported over long distances to hospitals where its decay product technetium-99m (with a half-life of only 6 hours, inconvenient for transport) is extracted and used for a variety of nuclear medicine procedures, where its short half-life is very useful.

Generators can also be constructed for supply of other daughter radioisotopes. Ruthenium 106 (Ru-106) is a commercially available radioisotope with a half-life of 668-373 days, making it a good candidate for a parent isotope in a cow or generator. The decay of Ru-106 to rhodium-106 (Rh-106) produces only a low energy beta of 39 Key that is not useful for therapy. However, Rh-106 has an energetic beta decay useful for brachytherapy: Rh-106 has a half-life of 30 seconds and decays by beta emission to palladium 106 (Pd-106) with a maximum decay energy of 3.541 Mev and an average energy of 96.9 Key. As an example, in some embodiments, the present invention features a device loaded from a Ruthenium-106 cow with an activity of rhodium-106 providing for the full prescribed dose. The device can be applied to the target volume to deliver the full activity of its contents. For example, the device may be placed over the target lesion for 10 half-lives (300 seconds), delivering all its radioactive energy and consuming the rhodium-106, depleting it to palladium.

In some embodiments, the present invention features the use of Ru-106 in secular equilibrium with Rh-106. Ru-106 decays by beta radiation to Rh-106. The two isotopes are in secular equilibrium with the decay rate of the combined source controlled by the Ru-106 parent but with the therapeutic beta radiations emanating from the daughter Rh-106.

Yttrium-90 is commercially available from Strontium-90 cows. As another example, in some embodiments, the present invention features the use of Yttrium-90 with a half-life of 64 hours. Y-90 decays to Zirconium 90 (Zr-90), a stable isotope, along three different routes via beta emission, wherein 99.985% of the time it decays with a maximum beta particle energy of 2.2801 MeV and a mean beta particle energy of 0.9337 MeV, or approximately or 1.5×10−13 joules. The other minor decay paths produce additional low energy gamma-rays, and electrons. Compared to the dominant path, the radiation doses from these paths are clinically negligible.

Currently, strontium-90 is also commercially available. As another example, in some embodiments, the present invention features the use of Strontium 90 (Sr-90) in secular equilibrium with Yttrium 90 (Y-90). Strontium 90 (Sr-90) decays by beta radiation to Yttrium 90 (Y-90). The parent Sr-90 isotope has a half-life of 28.79 years. The daughter Y-90 isotope has a half-life of 64.0 hours. The two isotopes are in secular equilibrium with the decay rate of the combined source controlled by the Sr-90 parent but with the therapeutic beta radiations emanating from the daughter Y-90 with maximum energy of 2.28 MeV and an average energy of 934 keV.

The Planning Target Volume (PTV) or Planning Treatment Volume (PTV) is a geometrical concept introduced for radiation treatment planning. The PTV is used to ensure that the prescribed dose is actually delivered to all parts of the target tissue. Without limiting the invention to any particular surgical practice, a medical journal article details the surgical creation of the bleb in which “the surgeon dissects backward with Westcott scissors to make a pocket approximately 10 to 15 mm posteriorly and sufficiently wide to accommodate the antimetabolite sponges”. In this example, the surgeon opened the potential space under the conjunctiva and Tenon's capsule creating an approximately 10 to 15 mm diameter bleb site. As an example, it would follow that the Target Volume could be defined as a disk of diameter 15 mm and depth of 0.3 mm, containing the conjunctiva and Tenon's capsule tissue.

For example, a prescription dose of brachytherapy of 10 Gray (1000 cGy) is 10 joules/kg absorbed dose throughout the Target Volume. Measurements have suggested a model Sr-90/Y-90 RBS with Activity of 1.48 GBq produces a surface dose rate of approximately 0.20 Gy per second. To deliver a dose of 10 Gy to the Target Volume would require an irradiation time of 50 seconds. The number nuclei that decay during this 50 second treatment would be 1.48×10⁹ Bq (disintegrations per second)×50 seconds=7.4×10¹⁰.

### Targets of the Eye

As previously discussed, the present invention provides methods and systems for applying beta radiation to a treatment area or target of the eye. In some embodiments, the target is a site of the bleb in an eye being treated for glaucoma with a MIGS implant or MIGS procedure. In some embodiments, the target is a site of the bleb in an eye treated with a trabeculectomy. In some embodiments, the target is a site of the bleb in an eye treated with minimally invasive micro sclerostomy (MIMS). In some embodiments, the target is a site of the hole in an eye treated with MIMS. In some embodiments, the target is a site of the implant that is surgically inserted into the eye for the purpose of treating glaucoma. In some embodiments, the target is a site of the eye associated with pterygium.

In some embodiments, the target comprises an entire bleb. In some embodiments, the target comprises a portion of a bleb. In some embodiments, the target area surrounds an end of the MIGS implant. In some embodiments, the target comprises at least a portion of the bleb above a drainage channel. In some embodiments, the target further comprises at least a portion of the bleb above a drainage channel and at least a portion of a perimeter of the bleb. In some embodiments, the target further comprises at least a portion of the bleb above a drainage channel, at least a portion of a perimeter of the bleb, and at least a portion of the bleb between the perimeter and the portion above the drainage channel.

In some embodiments, the target area is the entire bleb, e.g., the perimeter of the bleb, the center of the bleb, and the portions of the bleb in between the perimeter and the center. In some embodiments, the target area is the perimeter of the bleb, e.g., a ring-shaped target area. In some embodiments, the target is the perimeter of the bleb and a portion of the bleb next to the perimeter, e.g., the target may be annulus-shaped. In some embodiments, the target is a portion of the bleb in between the center and the perimeter. In some embodiments, the target is at least a portion of the center of the bleb. The present invention is not limited to the aforementioned descriptions of target areas. For example, in certain embodiments, the target is (or includes) tissue surrounding the rim of a drainage channel.

In some embodiments, the target is a target other than that associated with MIGS/MIMS/trabeculectomy. In some embodiments, the ophthalmic target is other targets than those associated with glaucoma drainage surgery. In some embodiments the target is inflammation, autoimmune mediated pathologies, or vascular pathologies of the eye. In some embodiments, the target comprises macrophages. In some embodiments, the target comprises fibroblasts. In some embodiments, the target comprises endothelial cells. In some embodiments, the target is associated with infections (for example, Herpes Simplex Keratitis or Tuberculous sclerokeratitis), Corneal ulcerations (for example, Moorens), Allergic disorders (for example, Vernal), benign or malignant Tumors (for example, Squamous Cell Carcinoma) or benign growths (for example, papillomas), Degenerations (for example, pterygium), Cicitarising disease (for example, pemphigoid), Inflammations (for example, meibomian gland), ocular manifestations of Stevens-Johnson syndrome, Drug-induced cicatrizing conjunctivitis, Ligneous conjunctivitis, Corneal Vascularization, Pterygia, Vernal Catarrh, Small papillomas of the eyelid, limbal carcinoma, ocular malignant melanoma, nevus pigmentosus of the conjunctiva, hemangioma, chalazion. In some embodiments, the target is in the orbit of the eye. The present invention includes other ophthalmic indications and is not limited to the aforementioned targets.

The system of the present invention delivers a dose of radiation to a target area or treatment area. The target area or treatment area may be a plane of a particular size (e.g., diameter) at a particular depth (e.g., a distance from the outer surface of the applicator, a distance from the surface of the eye, a distance from the top of the bleb, a distance from the RBS, etc.) within the tissue being exposed to beta radiation.

In certain embodiments, the target plane has a diameter of about 2 mm. In certain embodiments, the target plane has a diameter of about 3 mm. In certain embodiments, the target plane has a diameter of about 4 mm. In certain embodiments, the target plane has a diameter of about 5 mm. In certain embodiments, the target plane has a diameter of about 6 mm. In certain embodiments, the target plane has a diameter of about 7 mm. In certain embodiments, the target plane has a diameter of about 8 mm. In certain embodiments, the target plane has a diameter of about 9 mm. In certain embodiments, the target plane has a diameter of about 10 mm. In certain embodiments, the target plane has a diameter of about 11 mm. In certain embodiments, the target plane has a diameter of about 12 mm. In certain embodiments, the target plane has a diameter from 10 to 14 mm. In certain embodiments, the target plane has a diameter from 6 to 10 mm. In certain embodiments, the target plane has a diameter from 5 to 12 mm. In certain embodiments, the target plane has a diameter from 6 to 12 mm. In certain embodiments, the target plane has a diameter from 8 to 10 mm. In certain embodiments, the target plane has a diameter from 8 to 12 mm. In certain embodiments, the target plane has a diameter from 6 to 8 mm. In certain embodiments, the target plane has a diameter from 7 to 10 mm. In certain embodiments, the target plane has a diameter from 8 to 11 mm. In certain embodiments, the target plane has a diameter from 9 to 11 mm. In certain embodiments, the target plane has a diameter from 9 to 12 mm. The present invention is not limited to the aforementioned dimensions of the target surface.

In certain embodiments, the target plane is a distance from 0 to 700 microns, e.g., from the outer surface of the applicator (e.g., portion of the applicator that contacts the eye tissue), from the surface of the eye, from the top of the bleb, from the RBS, etc. In certain embodiments, the target plane is a distance from 0 to 100 microns, e.g., from the outer surface of the applicator (e.g., portion of the applicator that contacts the eye tissue), from the surface of the eye, from the top of the bleb, from the RBS, etc. In certain embodiments, the target plane is a distance from 100 to 200 microns, e.g., from the outer surface of the applicator (e.g., portion of the applicator that contacts the eye tissue), from the surface of the eye, from the top of the bleb, from the RBS, etc. In certain embodiments, the target plane is a distance from 200 to 400 microns, e.g., from the outer surface of the applicator (e.g., portion of the applicator that contacts the eye tissue), from the surface of the eye, from the top of the bleb, from the RBS, etc. In certain embodiments, the target plane is a distance from 200 to 600 microns, e.g., from the outer surface of the applicator (e.g., portion of the applicator that contacts the eye tissue), from the surface of the eye, from the top of the bleb, from the RBS, etc. In certain embodiments, the target plane is a distance from 400 to 600 microns, e.g., from the outer surface of the applicator (e.g., portion of the applicator that contacts the eye tissue), from the surface of the eye, from the top of the bleb, from the RBS, etc.

In certain embodiments, the dose across the particular target plane on or within the target varies by no more than 10% of the maximum dose. In certain embodiments, the dose across the particular plane on or within the target varies by no more than 15% of the maximum dose. In certain embodiments, the dose across the particular plane on or within the target varies by no more than 20% of the maximum dose. In certain embodiments, the dose across the particular plane on or within the target varies by no more than 30% of the maximum dose. In certain embodiments, the dose at any point on the target plane of the treatment area is within 10% of a dose at any other point on the target plane of the treatment area. In certain embodiments, the dose at any point on the target plane of the treatment area is within 20% of a dose at any other point on the target plane of the treatment area. In certain embodiments, the dose at any point on the target plane of the treatment area is within 30% of a dose at any other point on the target plane of the treatment area. In certain embodiments, the dose at any point on the target plane of the treatment area is within 40% of a dose at any other point on the target plane of the treatment area. In certain embodiments, the dose at any point on the target plane of the treatment area is within 50% of a dose at any other point on the target plane of the treatment area.

In some embodiments, a dose of radiation is delivered to a plurality of points on the target plane, wherein a dose received by one point on the target plane is within 50% of the dose received by any other point on the target plane. In some embodiments, a dose of radiation is delivered to a plurality of points on the target plane, wherein a dose received by one point on the target plane is within 40% of the dose received by any other point on the target plane. In some embodiments, a dose of radiation is delivered to a plurality of points on the target plane, wherein a dose received by one point on the target plane is within 30% of the dose received by any other point on the target plane. In some embodiments, a dose of radiation is delivered to a plurality of points on the target plane, wherein a dose received by one point on the target plane is within 20% of the dose received by any other point on the target plane. In some embodiments, a dose of radiation is delivered to a plurality of points on the target plane, wherein a dose received by one point on the target plane is within 15% of the dose received by any other point on the target plane. In some embodiments, a dose of radiation is delivered to a plurality of points on the target plane, wherein a dose received by one point on the target plane is within 10% of the dose received by any other point on the target plane.

### Application of Beta Radiation

The methods and systems of the present invention deliver a particular radiation dose to the target, e.g., to a plane within the target (e.g., a plane of a certain size at a certain depth representing a portion of the treatment area (e.g., PTV)).

In some embodiments, the methods and systems deliver a radiation dose of 1000 cGy (10Gy) to the target. In some embodiments, the methods and systems deliver a radiation dose of 900 cGy to the target. In some embodiments, the methods and systems deliver a radiation dose of 800 cGy to the target. In some embodiments, the methods and systems deliver a radiation dose of 750 cGy to the target. In some embodiments, the methods and systems deliver a radiation dose of 600 cGy to the target. In some embodiments, the methods and systems deliver a radiation dose of 500 cGy to the target. In some embodiments, the methods and systems deliver a radiation dose of 400 cGy to the target. In some embodiments, the methods and systems deliver a radiation dose of 300 cGy to the target. In some embodiments, the methods and systems deliver a radiation dose of 200 cGy to the target. In some embodiments, the methods and systems deliver a radiation dose of 100 cGy to the target. In some embodiments, the methods and systems deliver a radiation dose of 50 cGy to the target. In some embodiments, the methods and systems deliver a radiation dose of 1100 cGy to the target. In some embodiments, the methods and systems deliver a radiation dose of 1200 cGy to the target. In some embodiments, the methods and systems deliver a radiation dose of 1300 cGy to the target. In some embodiments, the methods and systems deliver a radiation dose of 1500 cGy to the target. In some embodiments, the methods and systems deliver a radiation dose from 600 cGy and 1500 cGy to the target. In some embodiments, the methods and systems deliver a radiation dose from 50 cGy to 100 cGy. In some embodiments, the methods and systems deliver a radiation dose from 100 cGy to 150 cGy. In some embodiments, the methods and systems deliver a radiation dose from 150 cGy to 200 cGy. In some embodiments, the methods and systems deliver a radiation dose from 200 cGy to 250 cGy. In some embodiments, the methods and systems deliver a radiation dose from 250 cGy to 300 cGy. In some embodiments, the methods and systems deliver a radiation dose from 300 cGy to 350 cGy. In some embodiments, the methods and systems deliver a radiation dose from 350 cGy to 400 cGy. In some embodiments, the methods and systems deliver a radiation dose from 400 cGy to 450 cGy. In some embodiments, the methods and systems deliver a radiation dose from 450 cGy to 500 cGy. In some embodiments, the methods and systems deliver a radiation dose from 500 cGy to 550 cGy. In some embodiments, the methods and systems deliver a radiation dose from 550 cGy to 600 cGy. In some embodiments, the methods and systems deliver a radiation dose from 600 cGy to 650 cGy. In some embodiments, the methods and systems deliver a radiation dose from 650 cGy to 700 cGy. In some embodiments, the methods and systems deliver a radiation dose from 700 cGy to 750 cGy. In some embodiments, the methods and systems deliver a radiation dose from 750 cGy to 800 cGy. In some embodiments, the methods and systems deliver a radiation dose from 800 cGy to 850 cGy. In some embodiments, the methods and systems deliver a radiation dose from 850 cGy to 900 cGy. In some embodiments, the methods and systems deliver a radiation dose from 900 cGy to 950 cGy. In some embodiments, the methods and systems deliver a radiation dose from 950 cGy to 1000 cGy. In some embodiments, the methods and systems deliver a radiation dose from 1000 cGy to 1050 cGy. In some embodiments, the methods and systems deliver a radiation dose from 1050 cGy to 1100 cGy. In some embodiments, the methods and systems deliver a radiation dose from 1100 cGy to 1150 cGy. In some embodiments, the methods and systems deliver a radiation dose from 1150 cGy to 1200 cGy. In some embodiments, the methods and systems deliver a radiation dose from 1200 cGy to 1250 cGy. In some embodiments, the methods and systems deliver a radiation dose from 1250 cGy to 1300 cGy. In some embodiments, the methods and systems deliver a radiation dose from 1300 cGy to 1350 cGy. In some embodiments, the methods and systems deliver a radiation dose from 1350 cGy to 1400 cGy. In some embodiments, the methods and systems deliver a radiation dose from 1400 cGy to 1450 cGy. In some embodiments, the methods and systems deliver a radiation dose from 1450 cGy to 1500 cGy. In some embodiments, the methods and systems deliver a radiation dose from 1500 cGy to 1550 cGy. In some embodiments, the methods and systems deliver a radiation dose from 1550 cGy to 1600 cGy. In some embodiments, the methods and systems deliver a radiation dose from 1600 cGy to 1800 cGy. In some embodiments, the methods and systems deliver a radiation dose from 1800 cGy to 2000 cGy. In some embodiments, the methods and systems deliver a radiation dose of 600, 650, 700, 750, 800, 850, 900, 950, 1000, 1050, 1100, 1150, 1200, 1250, 1300, 1350, 1400, 1450, or 1500 cGy to the target. In some embodiments, the methods and systems deliver a radiation dose of 1500 to 3200 cGy. In some embodiments, the methods and systems deliver a radiation dose of 3200 to 8000 cGy. In some embodiments, the methods and systems deliver a radiation dose of 8000 cGy to 10000 cGy. In some embodiments, the methods and systems deliver a radiation dose of greater than 10000 cGy.

The doses cited herein may refer to the doses at a particular depth from the surface of the device, for example at a depth of 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, etc.

In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 10% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 15% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 20% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 25% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 30% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 35% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 40% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 45% of a dose at any other point on the target. In some embodiments, the methods and systems provide a dose of beta radiation to the target (e.g., a plane of a particular size/diameter within the treatment area), wherein the dose at any point on the target (e.g., a plane of a particular size/diameter within the treatment area) is within 50% of a dose at any other point on the target.

In some embodiments, the methods and systems deliver the prescribed dose in a time from 10 seconds to 20 minutes. In some embodiments, the methods and systems deliver the prescribed dose in a time from 20 seconds to 10 minutes. In some embodiments, the methods and systems deliver the prescribed dose in a time from 20 seconds to 60 seconds. In some embodiments, the methods and systems deliver the prescribed dose in a time from 30 seconds to 90 seconds. In some embodiments, the methods and systems deliver the prescribed dose in a time from 60 seconds to 90 seconds. In some embodiments, the methods and systems deliver the prescribed dose in a time from 90 seconds to 2 minutes. In some embodiments, the methods and systems deliver the prescribed dose in a time from 2 minutes to 3 minutes.

In some embodiments, the methods and systems deliver the prescribed dose in a time from 3 minutes to 4 minutes. In some embodiments, the methods and systems deliver the prescribed dose in a time from 3 minutes to 5 minutes. In some embodiments, the methods and systems deliver the prescribed dose in a time from 3 minutes to 6 minutes. In some embodiments, the methods and systems deliver the prescribed dose in a time from 4 minutes to 5 minutes. In some embodiments, the methods and systems deliver the prescribed dose in a time from 4 minutes to 6 minutes. In some embodiments, the methods and systems deliver the prescribed dose in a time from 5 minutes to 6 minutes. In some embodiments, the methods and systems deliver the prescribed dose in a time from 6 minutes to 7 minutes. In some embodiments, the methods and systems deliver the prescribed dose in a time from 7 minutes to 8 minutes. In some embodiments, the methods and systems deliver the prescribed dose in a time from 8 minutes to 9 minutes. In some embodiments, the methods and systems deliver the prescribed dose in a time from 9 minutes to 10 minutes. In some embodiments, the methods and systems deliver the prescribed dose in a time from 10 minutes to 12 minutes. In some embodiments, the methods and systems deliver the prescribed dose in a time from 12 minutes to 15 minutes. In some embodiments, the methods and systems deliver the prescribed dose in a time from 15 minutes to 20 minutes.

In some embodiments, the methods and systems deliver the prescribed dose within 5 seconds. In some embodiments, the methods and systems deliver the prescribed dose within 10 seconds. In some embodiments, the methods and systems deliver the prescribed dose within 15 seconds. In some embodiments, the methods and systems deliver the prescribed dose within 20 seconds. In some embodiments, the methods and systems deliver the prescribed dose within 25 seconds. In some embodiments, the methods and systems deliver the prescribed dose within 45 seconds. In some embodiments, the methods and systems deliver the prescribed dose within 60 seconds. In some embodiments, the methods and systems deliver the prescribed dose within 90 seconds. In some embodiments, the methods and systems deliver the prescribed dose within 2 minutes. In some embodiments, the methods and systems deliver the prescribed dose within 3 minutes. In some embodiments, the methods and systems deliver the prescribed dose within 4 minutes. In some embodiments, the methods and systems deliver the prescribed dose within 5 minutes. In some embodiments, the methods and systems deliver the prescribed dose within 6 minutes. In some embodiments, the methods and systems deliver the prescribed dose within 7 minutes. In some embodiments, the methods and systems deliver the prescribed dose within 8 minutes. In some embodiments, the methods and systems deliver the prescribed dose within 9 minutes. In some embodiments, the methods and systems deliver the prescribed dose within 10 minutes. In some embodiments, the methods and systems deliver the prescribed dose within 11 minutes. In some embodiments, the methods and systems deliver the prescribed dose within 12 minutes. In some embodiments, the methods and systems deliver the prescribed dose within 13 minutes. In some embodiments, the methods and systems deliver the prescribed dose within 14 minutes. In some embodiments, the methods and systems deliver the prescribed dose within 15 minutes. In some embodiments, the methods and systems deliver the prescribed dose within 16 minutes. In some embodiments, the methods and systems deliver the prescribed dose within 17 minutes. In some embodiments, the methods and systems deliver the prescribed dose within 18 minutes. In some embodiments, the methods and systems deliver the prescribed dose within 19 minutes. In some embodiments, the methods and systems deliver the prescribed dose within 20 minutes. In some embodiments, the methods and systems deliver the prescribed dose in a time frame greater than 20 minutes.

In some embodiments, a dose (e.g., a prescribed dose) may be delivered in a single application. In other embodiments, a dose (e.g., a prescribed dose) may be fractionated and applied in multiple applications. For example, in some embodiments, radiation (e.g., a prescribed dose) may be applied over the course of 2 applications. In some embodiments, radiation (e.g., a prescribed dose) may be applied over the course of 3 applications. In some embodiments, radiation (e.g., a prescribed dose) may be applied over the course of 4 applications. In some embodiments, radiation (e.g., a prescribed dose) may be applied over the course of 5 applications. In some embodiments, radiation (e.g., a prescribed dose) may be applied over the course of more than 5 applications. In some embodiments, radiation (e.g., a prescribed dose) may be applied over the course of 20 applications. In some embodiments, radiation (e.g., a prescribed dose) may be applied over the course of more than 20 applications.

Each application may deliver an equal sub-dose. In some embodiments, one or more of the sub-doses are different. For example, one or more of the sub-doses may be different so as to increase or decrease with each additional application.

According to one embodiment, a dose of radiation may be applied prior to the treatment procedure, e.g., surgery for implantation of a device, e.g., MIGS device, or other appropriate glaucoma procedure, e.g., MIMS. For example, in some embodiments, a dose of radiation may be applied one or more days prior to a surgery (e.g., insertion of a device, MIMS, etc.). In some embodiments, a dose of radiation may be applied within a 24-hour prior before a surgery (e.g., insertion of a device). In some embodiments, a dose of radiation may be applied just prior to a surgery (e.g., insertion of a device, MIMS, etc.), e.g., 1 hour before, 30 minutes before, 15 minutes before, 5 minutes before 1 minute before, etc. In some embodiments, a dose of radiation may be applied during a procedure, e.g., for implantation of a device. In some embodiments, a dose of radiation may be applied right after a surgery (e.g., implantation of a device (e.g., MIGS device), MIMS, etc.), e.g., within 1 minute, 2 minutes, 3 minutes, 5 minutes, 10 minutes, etc.). In some embodiments, a dose of radiation may be applied before an incision is made into the conjunctiva. In some embodiments, a dose of radiation may be applied after an incision is made into the conjunctiva. In other embodiments, a dose of radiation may be applied after a surgery (e.g., insertion of a device). In some embodiments, a dose of radiation may be applied within a 24-hour period after a surgery (e.g., insertion of a device). In some embodiments, a dose of radiation may be applied within one to two days after a surgery (e.g., insertion of a device). In some embodiments, a dose of radiation may be applied within 2 or more days after a surgery (e.g., insertion of a device). In some embodiments the dose may be applied any time after the glaucoma surgery. In some embodiments, the dose is applied months or years after the glaucoma surgery. For example, a dose may be given to patients that did not receive a dose during surgery but at a future date have scar or needling procedures to break up scar tissue.

### Methods

The present invention features methods and systems for applying a therapeutic dose of beta radiation to a treatment area, such as a target area of a bleb for draining aqueous humor, such as but not limited to a bleb associated with a Minimally Invasive Glaucoma Surgery (MIGS) implant or foreign body inserted between an anterior chamber of the eye and a subconjunctival space of the eye or between the anterior chamber of the eye and a space between the conjunctiva and Tenon's capsule, in combination with glaucoma surgery. The methods and systems herein may be used to apply beta radiation to a target area in the eye to help maintain functioning blebs and/or drainage holes arising from glaucoma drainage procedures or surgeries, to help avoid scar formation or wound reversion, to inhibit or reduce fibrogenesis and/or inflammation in the blebs or surrounding areas, to treat glaucoma, to reduce intraocular pressure (IOP), to achieve and/or maintain a healthy IOP, for causing cell cycle arrest in fibroblasts on the Tenon's capsule, to enhance function of a drainage device such as a MIGS implant, etc. The present invention is not limited to the applications disclosed herein.

The methods may feature the application of the therapeutic dose of beta radiation from a radioisotope to a target area of the eye using an applicator system.

In some embodiments, the methods comprise performing glaucoma surgery, which forms a bleb for draining aqueous humor. For example, the method may comprise implanting a Minimally Invasive Glaucoma Surgery (MIGS) implant within the eye, wherein the implant causes formation of a bleb (e.g., in the subconjunctival space of the eye, in a space between the conjunctiva and Tenon's capsule); the bleb functions to drain aqueous humor. In certain embodiments, the implant is inserted trans-sclerally, between an anterior chamber of the eye and a subconjunctival space of the eye, between the anterior chamber of the eye and a space between the conjunctiva and Tenon's capsule, etc.

The methods feature applying a therapeutic dose of beta radiation to the target site (e.g., drainage site or other appropriate site) at or around the time of glaucoma surgery (e.g., implantation of a drainage device, e.g., MIGS implantation), e.g., before glaucoma surgery, after glaucoma surgery, etc. For example, the method may comprise applying the beta radiation prior to insertion of a MIGS implant, prior to incision of the conjunctive, prior to creation of a hole associated with MIMS, etc. In some embodiments, the method comprises applying the beta radiation after insertion of a MIGS implant, prior to incision of the conjunctive, prior to creation of a hole associated with MIMS, etc.

The methods herein may also feature applying a drug to the eye, e.g., to the target, to an area near the target, to a site of a drainage device or implant, to the side of the bleb, to a different part of the eye, etc. Non-limiting examples of drugs include mitomycin C, 5 fluorouracil, an anti-VEGF composition, and other appropriate compositions. In some embodiments, the drug is administered before, during, and/or after a surgical procedure.

As previously discussed, the beta radiation may be applied via a radionuclide brachytherapy source (RBS). The RBS may be applied to the target via an applicator. As previously discussed, in some embodiments, the beta radiation is Strontium-90 (Sr-90), Phosphorus-32 (P-32), Ruthenium 106 (Ru-106), Yttrium 90 (Y-90), or a combination thereof.

Examples of the present invention include but are not limited to a radioisotope that emits beta radiation for use in a method of treating glaucoma, a radioisotope that emits beta radiation for use for use in preventing or reducing scar formation in a drainage bleb or drainage channel in an eye being treated or having been treated with glaucoma surgery, a radioisotope that emits beta radiation for use for use in a method for reducing intraocular pressure (IOP) in an eye being treated or having been treated with glaucoma surgery, a composition comprising a source of beta radiation for use in a method for achieving a healthy intraocular pressure (IOP) in a human eye being treated or having been treated for glaucoma, etc. The radioisotope or composition may be administered to the eye such that beta radiation from the source of beta radiation is applied to a target area of the eye, wherein the target area is associated with the drainage bleb, a drainage channel, or a glaucoma drainage implant.

Examples of methods of the present invention include but are not limited to methods of treating glaucoma, methods of preventing or reducing scar formation in a drainage bleb or drainage channel in an eye being treated or having been treated with glaucoma surgery, methods for reducing intraocular pressure (IOP) in an eye being treated or having been treated with glaucoma surgery, methods for achieving a healthy intraocular pressure (IOP) in a human eye being treated or having been treated for glaucoma, etc.

The glaucoma surgery allows aqueous humor to drain into a bleb in a subconjunctival space or space between a conjunctiva and Tenon's capsule. In certain embodiments, the glaucoma surgery is Minimally Invasive Glaucoma Surgery (MIGS).

In certain embodiments, the methods herein are effective for one or a combination of: maintaining a functioning drainage bleb; inhibiting or reducing fibrogenesis and inflammation in the bleb, around the drainage implant, or around the drainage channel; and reducing conjunctival inflammation. In certain embodiments, the methods herein are effective for achieving a healthy IOP. In certain embodiments, the methods herein are effective for maintaining a healthy IOP. In certain embodiments, the methods herein are effective for lowering IOP and maintaining said IOP.

Inhibiting or reducing fibrogenesis and inflammation in the bleb may be measured according to a predetermined bleb grading scale. In certain embodiments, the predetermined bleb grading scale is Moorfields bleb grading scale (MBGS). In certain embodiments, the predetermined bleb grading scale is Indiana Bleb Appearance Grading Scale (IBAGS).

The methods herein feature applying a therapeutic dose of the beta radiation from a radioisotope or composition or source to a target area of the eye. The target area may be associated with the bleb. In certain embodiments, the target area is associated with a glaucoma drainage implant. In certain embodiments, the target area is associated with a drainage channel.

In certain embodiments, the methods herein further comprise administering a drug to the target area. Non-limiting examples of drugs include mitomycin C, 5 fluorouracil, an anti-VEGF composition, etc.

In certain embodiments, intraocular pressure (IOP) is reduced to 12 mmHg or less. In certain embodiments, IOP is reduced to 10 mmHg or less. In certain embodiments, IOP is reduced to from 5 to 10 mmHg. In certain embodiments, IOP is reduced to from 5 to 12 mmHg. In certain embodiments, IOP is reduced to from 8 to 10 mmHg. T In certain embodiments, IOP is reduced to from 8 to 12 mmHg.

In some embodiments, the method is effective for reducing IOP by 10% or more 6 months after treatment. In some embodiments, the method is effective for reducing IOP by 20% or more 6 months after treatment. In some embodiments, the method is effective for reducing IOP by 30% or more 6 months after treatment. In some embodiments, the method is effective for reducing IOP by 40% or more 6 months after treatment. In some embodiments, the method is effective for reducing IOP by 50% or more 6 months after treatment.

In some embodiments, the method is effective for reducing IOP by 10% or more 12 months after treatment. In some embodiments, the method is effective for reducing IOP by 20% or more 12 months after treatment. In some embodiments, the method is effective for reducing IOP by 30% or more 12 months after treatment. In some embodiments, the method is effective for reducing IOP by 40% or more 12 months after treatment. In some embodiments, the method is effective for reducing IOP by 50% or more 12 months after treatment.

In some embodiments, the method is effective for reducing IOP by 10% or more 24 months after treatment. In some embodiments, the method is effective for reducing IOP by 20% or more 24 months after treatment. In some embodiments, the method is effective for reducing IOP by 30% or more 24 months after treatment. In some embodiments, the method is effective for reducing IOP by 40% or more 24 months after treatment. In some embodiments, the method is effective for reducing IOP by 50% or more 24 months after treatment.

In some embodiments, the method is effective for reducing IOP by 10% or more 36 months after treatment. In some embodiments, the method is effective for reducing IOP by 20% or more 36 months after treatment. In some embodiments, the method is effective for reducing IOP by 30% or more 36 months after treatment. In some embodiments, the method is effective for reducing IOP by 40% or more 36 months after treatment. In some embodiments, the method is effective for reducing IOP by 50% or more 36 months after treatment.

In some embodiments, the method is effective for reduction of IOP and subsequent stabilization of IOP. In some embodiments, stabilization of IOP is wherein the IOP does not increase by more than 10% at 3 months after treatment. In some embodiments, stabilization of IOP is wherein the IOP does not increase by more than 10% at 6 months after treatment. In some embodiments, stabilization of IOP is wherein the IOP does not increase by more than 10% at 12 months after treatment. In some embodiments, stabilization of IOP is wherein the IOP does not increase by more than 10% at 24 months after treatment. In some embodiments, stabilization of IOP is wherein the IOP does not increase by more than 10% at 36 months after treatment.

In some embodiments, stabilization of IOP is wherein the IOP does not increase by more than 20% at 3 months after treatment. In some embodiments, stabilization of IOP is wherein the IOP does not increase by more than 20% at 6 months after treatment. In some embodiments, stabilization of IOP is wherein the IOP does not increase by more than 20% at 12 months after treatment. In some embodiments, stabilization of IOP is wherein the IOP does not increase by more than 20% at 24 months after treatment. In some embodiments, stabilization of IOP is wherein the IOP does not increase by more than 20% at 36 months after treatment.

In some embodiments, stabilization of IOP is wherein the IOP does not increase by more than 25% at 24 months after treatment. In some embodiments, stabilization of IOP is wherein the IOP does not increase by more than 25% at 36 months after treatment.

In some embodiments, the systems and devices of the present invention may be used for methods associated with needling procedures, e.g., procedures to the bleb to free or remove scar tissue and/or cystic structures in and/or around the bleb and/or surgery site that may later arise from wound healing or scarring or inflammatory responses to the glaucoma surgery. Needling procedures may affect surgical site morphology, restore the function of the surgery and/or lower the IOP.

### Brachytherapy System and Applicator

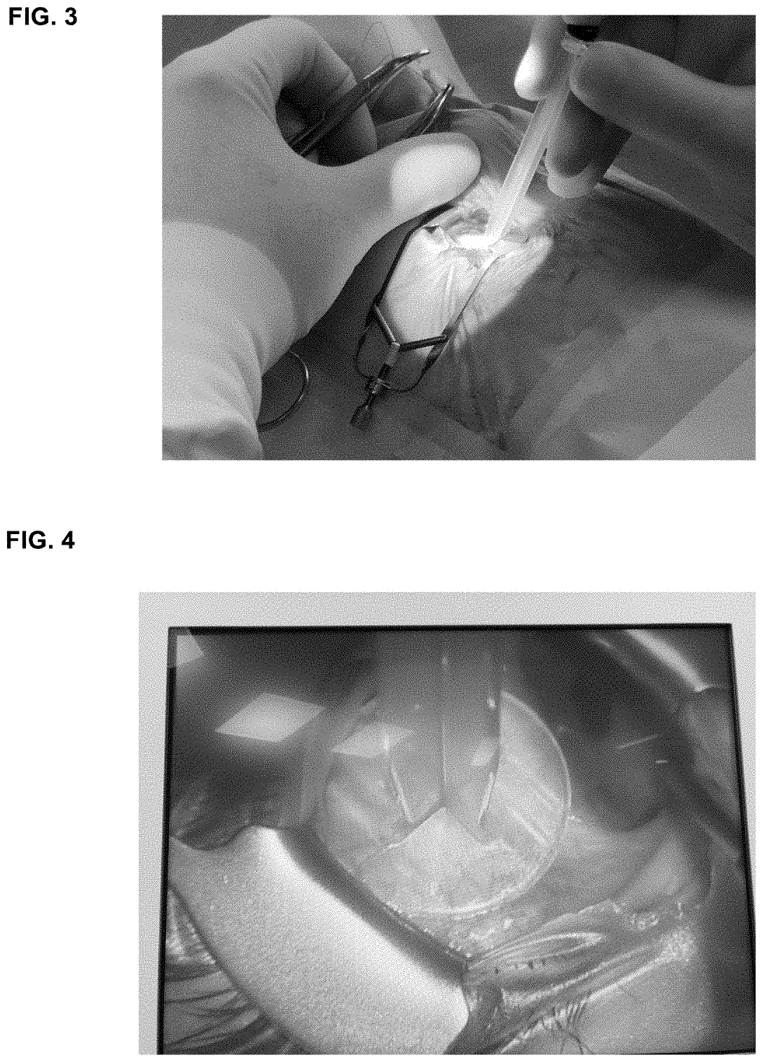

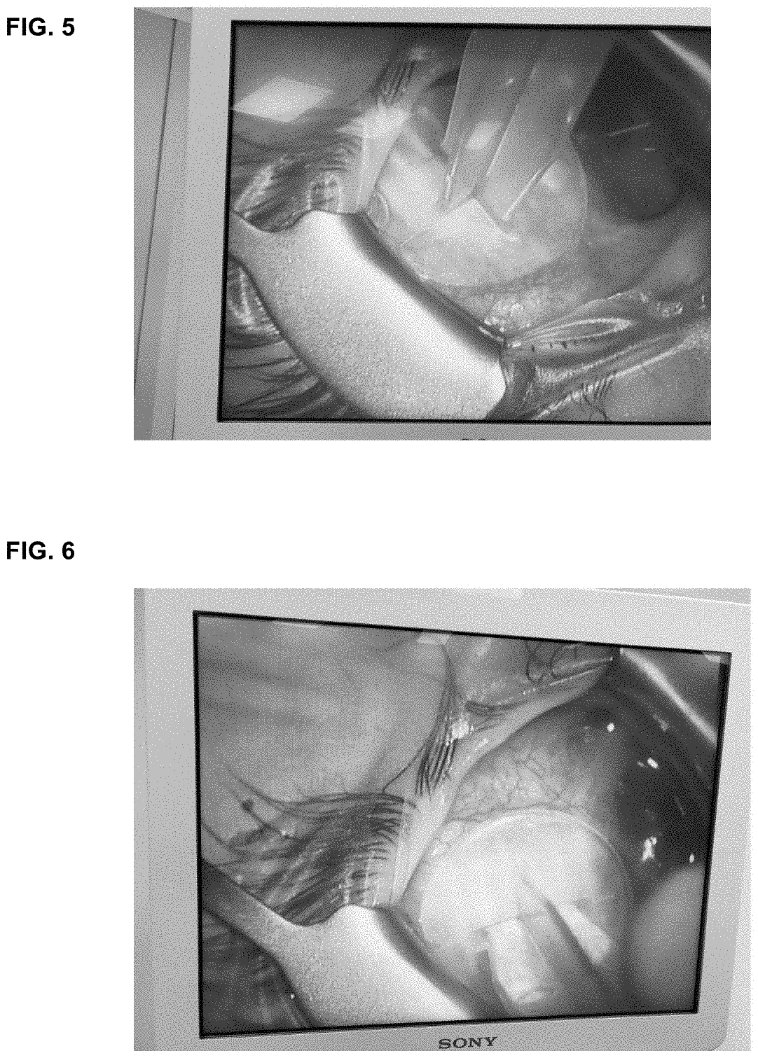

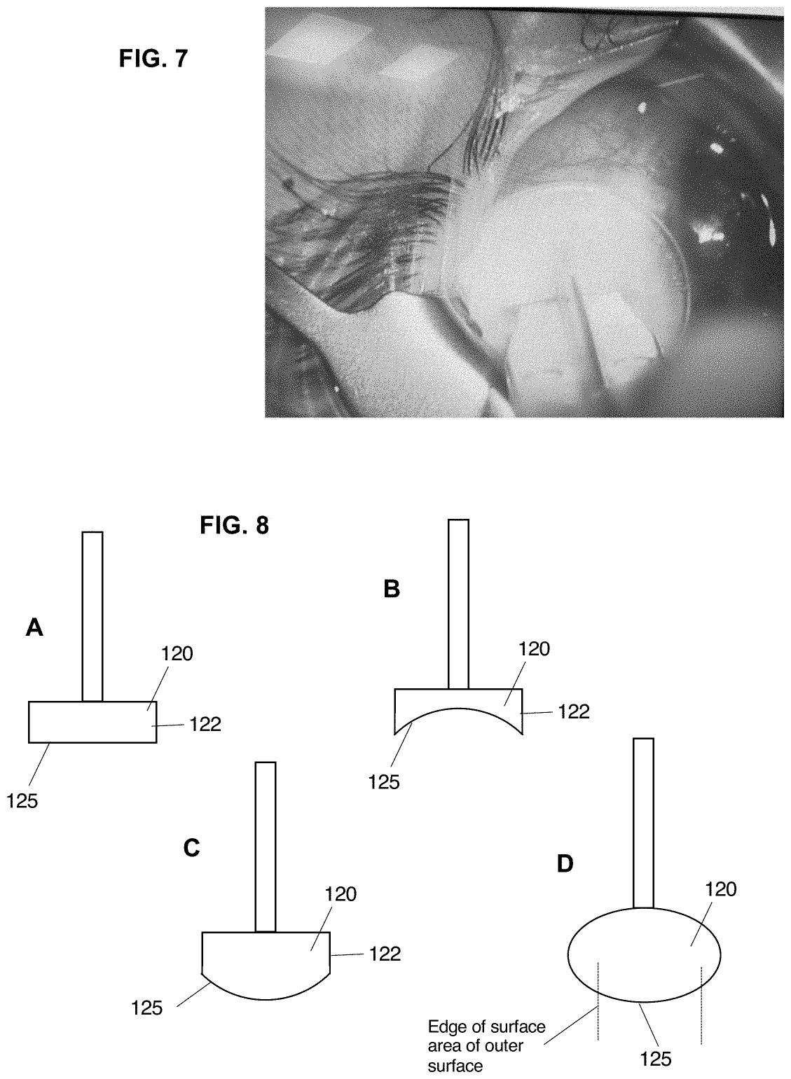

Referring to FIG. 3, FIG. 4, FIG. 5, FIG. 6, and FIG. 7, Inventors discovered that an applicator with a tip (distal end) with a radiation source could be placed in contact with the swollen conjunctiva of the eye and pressed such that all (or substantially all) of the fluid associated with the swelling can be evacuated, creating a uniform distance between the tip of the applicator and the bottom surface of the bleb. This was surprising because the bleb was thought to be too edematous for an applicator to push away all of the fluid therein, e.g., the conjunctiva edema fluid, the fluid between the tip of the applicator and the bottom of the bleb. FIG. 3, FIG. 4, FIG. 5, FIG. 6, and FIG. 7 show the progression of blanching as progressively more pressure is applied to the applicator system.

FIG. 8 shows non-limiting examples of applicator systems with distal ends (120). In certain embodiments, the outer surface (125) (e.g., the surface that contacts the eye) of the tip or distal end (120) of the applicator is flat. In some embodiments, the outer surface (125) (e.g., the surface that contacts the eye) of the tip or distal end (120) of the applicator has a curvature. For example, in certain embodiments, the curvature is a concave curvature. In certain embodiments, the curvature is a convex curvature. The present invention is not limited to the aforementioned shapes of outer surfaces.

In certain embodiments, the surface area of the outer surface (125) of the distal end (120) is the area from edge(s) to edge(s), the edges being the locations wherein the outer surface (125) intersects with a side wall (122). The edges and/or side walls may be well defined, as shown in examples A, B, and C in FIG. 8. In certain embodiments, the edges may be defined as the locations where the radius (or radii) of curvature changes. In some embodiments, the side walls may not be well defined, e.g., as shown in example D in FIG. 8. In certain embodiments, the surface area of the outer surface (125) of the distal end (120) is the area through with the beta radiation is emitted to achieve the therapeutic dose throughout the target area. For example, the dashed lines shown on Example D in FIG. 8 define the surface area of the outer surface (125) of the distal end (120). The dashed lines may represent the boundaries of the outer surface (125) that provides the therapeutic dose of radiation to the target area.

In certain embodiments, the distal end or tip has a curvature with a radius of curvature from near 0 to about 120 mm to flat, e.g., the radius of curvature of the eye. The present invention is not limited to surfaces with radii of curvature from 0 to 120 mm to flat, e.g., the radius of curvature may be from 100 to 120 mm, 120 to 150 mm, 150 to 180 mm, 120 mm to 1,000 mm, 120 mm to 10,000 mm, etc. The outer surface may comprise surfaces with different radii of curvature, e.g., a portion of the outer surface may be flat and a portion may be curved; a portion of the outer surface may have one radius of curvature and a second portion may have a second (different) radius of curvature, etc.

In certain embodiments, the shape of the outer surface may be a way of shaping the radiation, e.g., the outer surface may have a particular curvature (or lack thereof) as a mechanism for helping provide a specific radiation dose profile.

As previously discussed, methods using the applicator systems herein include but are not limited to methods for inhibiting or reducing fibrogenesis and inflammation in a bleb of an eye being treated for glaucoma. The present invention also provides methods for maintaining a functioning drainage bleb in the eye of a patient being treated for glaucoma. The present invention also provides methods for treating glaucoma. The present invention also provides methods for reducing intraocular pressure (IOP) in an eye. The present invention also provides methods for reducing inflammation in an eye having a foreign body therein, the foreign body being a Minimally Invasive Glaucoma Surgery (MIGS) implant inserted between an anterior chamber of the eye and a subconjunctival space of the eye or between the anterior chamber of the eye and a space between the conjunctiva and Tenon's capsule (the implant causes formation of a bleb for draining aqueous humor).

As an example, the present invention features a method of inhibiting or reducing fibrogenesis and inflammation in a bleb of an eye or a patient being treated or having been treated for glaucoma, a method of maintaining a functioning drainage bleb in the eye of a patient being treated or having been treated for glaucoma, a method of treating glaucoma, a method for reducing intraocular pressure, a method of reducing inflammation in an eye having a foreign body therein (e.g., a Minimally Invasive Glaucoma Surgery (MIGS) implant inserted between an anterior chamber of the eye and a subconjunctival space of the eye or between the anterior chamber of the eye and a space between the conjunctiva and Tenon's capsule, wherein the implant causes formation of a bleb for draining aqueous humor), etc.

Referring to any of the embodiments herein, in some embodiments, the method comprises applying a therapeutic dose of beta radiation from a radioisotope to a target area of the eye using an applicator system. The applicator system may comprise a handle and a distal end with a radioisotope embedded or engaged therein, wherein the distal end has an outer surface for contacting the eye. The method may comprise placing the outer surface of the distal end of the applicator system in contact with the eye at the target area and pressing upon the applicator system such that at least a portion of conjunctiva edema fluid is pushed away from the target area. In some embodiments, the method comprises placing the applicator system in contact with the eye at the target area and pressed upon the applicator system, wherein the distance from the outer surface of the distal end of the applicator system and the bottom surface of the bleb is substantially uniform across the target area.

In certain embodiments, the beta radiation causes cell cycle arrest in fibroblasts on the Tenon's capsule to inhibit or reduce the fibrotic process and inflammation that leads to bleb failure. In certain embodiments, the method is effective to maintain the drainage function of the bleb. In certain embodiments, the method is effective for reducing an Intraocular Pressure (IOP) of the eye. In certain embodiments, the method is effective for reducing an Intraocular Pressure (IOP) of the eye. In certain embodiments, the method is effective for reducing inflammation caused by the presence of the foreign body.

Non-limiting examples of targets or treatment areas are described herein. The target may be, but is not limited to, at least a portion of the bleb. In some embodiments, the target comprises an entire bleb. In some embodiments, the target comprises a portion of a bleb. In some embodiments, the target area surrounds an end of the MIGS implant. In some embodiments, the target comprises at least a portion of the bleb above a drainage channel. In some embodiments, the target further comprises at least a portion of the bleb above a drainage channel and at least a portion of a perimeter of the bleb. In some embodiments, the target further comprises at least a portion of the bleb above a drainage channel, at least a portion of a perimeter of the bleb, and at least a portion of the bleb between the perimeter and the portion above the drainage channel.

In some embodiments, the eye being treated for glaucoma has a Minimally Invasive Glaucoma Surgery (MIGS) implant inserted trans-sclerally causing formation of a bleb in the subconjunctival space of the eye or in a space between the conjunctiva and Tenon's capsule.

In some embodiments, the method comprises implanting a Minimally Invasive Glaucoma Surgery (MIGS) implant within the eye, wherein the implant is inserted trans-sclerally to cause formation of a bleb in the subconjunctival space of the eye or in a space between the conjunctiva and Tenon's capsule, the bleb functions to drain aqueous humor.

In some embodiments, the outer surface of the distal end of the applicator is flat. In some embodiments, the outer surface of the distal end of the applicator has curvature. In some embodiments, the curvature is a convex curvature. In some embodiments, the curvature is a concave curvature. In some embodiments, the outer surface of the distal end of the applicator has a portion that has curvature and a portion that is flat.

In some embodiments, when the applicator system is placed in contact with the eye at the target area and pressed upon, at least 25% of the surface area of the outer surface of the distal end is in contact with the eye. In some embodiments, when the applicator system is placed in contact with the eye at the target area and pressed upon, at least 50% of the surface area of the outer surface of the distal end is in contact with the eye. In some embodiments, when the applicator system is placed in contact with the eye at the target area and pressed upon, at least 75% of the surface area of the outer surface of the distal end is in contact with the eye. In some embodiments, when the applicator system is placed in contact with the eye at the target area and pressed upon, at least 80% of the surface area of the outer surface of the distal end is in contact with the eye. In some embodiments, when the applicator system is placed in contact with the eye at the target area and pressed upon, at least 90% of the surface area of the outer surface of the distal end is in contact with the eye. In some embodiments, when the applicator system is placed in contact with the eye at the target area and pressed upon, at least 95% of the surface area of the outer surface of the distal end is in contact with the eye. In some embodiments, when the applicator system is placed in contact with the eye at the target area and pressed upon, at least 99% of the surface area of the outer surface of the distal end is in contact with the eye.

In some embodiments, the radioisotope comprises Strontium-90 (Sr-90), Phosphorus-32 (P-32), Ruthenium 106 (Ru-106), Yttrium 90 (Y-90), or a combination thereof. In some embodiments, the therapeutic dose is from 500-1000 cGy. In some embodiments, the therapeutic dose is from 450-1050 cGy.

In some embodiments, the outer surface has a radius of curvature from 120 mm to flat. In some embodiments, the outer surface has a radius of curvature from 120 mm to 1,000 mm. In some embodiments, the outer surface of the distal end is 12 mm in diameter. In some embodiments, the outer surface of the distal end is from 8 to 10 mm in diameter. In some embodiments, the outer surface of the distal end is from 10 to 12 mm in diameter. In some embodiments, the outer surface of the distal end is from 6 to 14 mm in diameter. In some embodiments, the outer surface of the distal end is from 7 to 14 mm in diameter. Note the geometry of the outer surface of the applicator system is not limited to a circular geometry. Any appropriate geometry that achieves the therapeutic dose profile may be considered.

As previously discussed, with reference to any of the methods, systems, and compositions herein, in some embodiments, the methods further comprise administering a drug to the eye, e.g., to the target area. As a non-limiting example, the methods may further comprise administering pharmaceutical eyedrops or a liquid anti-metabolite. In various embodiments, the drug may be administered before, during, or after the surgical implantation procedure.

In some embodiments, the step of pressing the outer surface of the distal end of the applicator system causes blanching of tissue underneath the outer surface.

As previously discussed, the present invention provides a brachytherapy system for applying beta radiation to a target of the eye, the target being a site of a bleb in an eye being treated for glaucoma. The brachytherapy system comprises a radionuclide brachytherapy source (RBS) for supplying the beta radiation that is delivered to the target.

The RBS of the present invention is constructed in a manner that is consistent with the Federal Code of Regulations, but is not limited to the terms mentioned in the Code. For example, the RBS of the present invention may further comprise a substrate. Also, for example, in addition to being enclosed by the mentioned “gold, titanium, stainless steel, or platinum”, in some embodiments the radionuclide (isotope) of the present invention may be enclosed by a combination of one or more of “gold, titanium, stainless steel, or platinum”. In some embodiments, the radionuclide (isotope) of the present invention may be enclosed by one or more layers of an inert material comprising silver, gold, titanium, stainless steel, platinum, tin, zinc, nickel, copper, other metals, ceramics, glass, or a combination of these.

In some embodiments, the RBS comprises a substrate, a radioactive isotope (e.g., Sr-90, Y-90, Rh-106, P-32, etc.), and an encapsulation. In some embodiments, the isotope is coated on the substrate, and both the substrate and isotope are further coated with the encapsulation. In some embodiments, the radioactive isotope is embedded in the substrate. In some embodiments, the radioactive isotope is part of the substrate matrix. In some embodiments, the encapsulation may be coated onto the isotope, and optionally, a portion of the substrate. In some embodiments, the encapsulation is coated around the entire substrate and the isotope. In some embodiments, the encapsulation encloses the isotope. In some embodiments, the encapsulation encloses the entire substrate and the isotope. In some embodiments, the radioactive isotope is an independent piece and is sandwiched between the encapsulation and the substrate.

In some embodiments, a surface on the substrate is shaped in a manner to provide a controlled projection of radiation. The substrate may be constructed from a variety of materials. For example, in some embodiments the substrate is constructed from a material comprising, a silver, an aluminum, a stainless steel, tungsten, nickel, tin, zirconium, zinc, copper, a metallic material, a ceramic material, a ceramic matrix, the like, or a combination thereof. In some embodiments, the substrate functions to shield a portion of the radiation emitted from the isotope. The encapsulation may be constructed from a variety of materials, for example from one or more layers of an inert material comprising a steel, a silver, a gold, a titanium, a platinum, another bio-compatible material, the like, or a combination thereof.

The radionuclide brachytherapy source (RBS) is constructed to provide a substantially uniform radiation dose across the target. Previous radiation applicators may only treat the center part of the target or under-dose the peripheral area and/or overdose the center. The present invention may provide a more uniform dose across the target area.

In some embodiments, the RBS has a diameter from 4 to 20 mm. In some embodiments, the RBS has a diameter from 5 to 15 mm. In some embodiments, the RBS has a diameter from 10 to 20 mm. In some embodiments, the RBS has a diameter from 10 to 15 m. In some embodiments, the RBS has a diameter from 5 to 7 mm (e.g., 5 mm, 6 mm, 7 mm). In some embodiments, the RBS has a diameter from 7 to 10 mm (e.g., 7 mm, 7.5 mm, 8 mm, 8.5 mm, 9 mm, 9.5 mm, 10 mm). In some embodiments, the RBS has a diameter from 9 to 12 mm (e.g., 9 mm, 9.5 mm, 10 mm, 10.5 mm, 11 mm, 11.5 mm, 12 mm). In some embodiments, the RBS has a diameter from 10 to 14 mm (e.g., 10 mm, 10.5 mm, 11 mm, 11.5 mm, 12 mm, 12.5 mm, 13 mm, 13.5 mm, 14 mm). In some embodiments, the RBS has a diameter from 12 to 16 mm (e.g., 12 mm, 12.5 mm, 13 mm, 13.5 mm, 14 mm, 14.5 mm, 15 mm, 15.5 mm, 16 mm). In some embodiments, the RBS has a diameter from 14 to 18 mm (e.g., 14 mm, 14.5 mm, 15 mm, 15.5 mm, 16 mm, 16.5 mm, 17 mm, 17.5 mm, 18 mm). In some embodiments, the RBS has a diameter of 3 mm. In some embodiments, the RBS has a diameter of 4 mm. In some embodiments, the RBS has a diameter of 5 mm. In some embodiments, the RBS has a diameter of 5 mm. In some embodiments, the RBS has a diameter of 6 mm. In some embodiments, the RBS has a diameter of 7 mm. In some embodiments, the RBS has a diameter of 8 mm. In some embodiments, the RBS has a diameter of 9 mm. In some embodiments, the RBS has a diameter of 10 mm. In some embodiments, the RBS has a diameter of 11 mm. In some embodiments, the RBS has a diameter of 12 mm. In some embodiments, the RBS has a diameter of 13 mm. In some embodiments, the RBS has a diameter of 14 mm. In some embodiments, the RBS has a diameter of 15 mm. In some embodiments, the RBS has a diameter of 16 mm. In some embodiments, the RBS has a diameter of 17 mm. In some embodiments, the RBS has a diameter of 18 mm. In some embodiments, the RBS has a diameter of 19 mm. In some embodiments, the RBS has a diameter of 20 mm. In some embodiments, the RBS has a diameter of more than 20 mm.

In some embodiments, the RBS delivers a radiation dose of 1000 cGy (10Gy) to the target. In some embodiments, the RBS delivers a radiation dose of 900 cGy to the target. In some embodiments, the RBS delivers a radiation dose of 800 cGy to the target. In some embodiments, the RBS delivers a radiation dose of 750 cGy to the target. In some embodiments, the RBS delivers a radiation dose of 600 cGy to the target. In some embodiments, the RBS delivers a radiation dose of 500 cGy to the target. In some embodiments, the RBS delivers a radiation dose of 400 cGy to the target. In some embodiments, the RBS delivers a radiation dose of 300 cGy to the target. In some embodiments, the RBS delivers a radiation dose of 200 cGy to the target. In some embodiments, the RBS delivers a radiation dose of 100 cGy to the target. In some embodiments, the RBS delivers a radiation dose of 50 cGy to the target. In some embodiments, the RBS delivers a radiation dose of 1100 cGy to the target. In some embodiments, the RBS delivers a radiation dose of 1200 cGy to the target. In some embodiments, the RBS delivers a radiation dose of 1300 cGy to the target. In some embodiments, the RBS delivers a radiation dose of 1500 cGy to the target. In some embodiments, the RBS delivers a radiation dose from 600 cGy and 1500 cGy to the target. In some embodiments, the RBS delivers a radiation dose from 50 cGy to 100 cGy. In some embodiments, the RBS delivers a radiation dose from 100 cGy to 150 cGy. In some embodiments, the RBS delivers a radiation dose from 150 cGy to 200 cGy. In some embodiments, the RBS delivers a radiation dose from 200 cGy to 250 cGy. In some embodiments, the RBS delivers a radiation dose from 250 cGy to 300 cGy. In some embodiments, the RBS delivers a radiation dose from 300 cGy to 350 cGy. In some embodiments, the RBS delivers a radiation dose from 350 cGy to 400 cGy. In some embodiments, the RBS delivers a radiation dose from 400 cGy to 450 cGy. In some embodiments, the RBS delivers a radiation dose from 450 cGy to 500 cGy. In some embodiments, the RBS delivers a radiation dose from 500 cGy to 550 cGy. In some embodiments, the RBS delivers a radiation dose from 550 cGy to 600 cGy. In some embodiments, the RBS delivers a radiation dose from 600 cGy to 650 cGy. In some embodiments, the RBS delivers a radiation dose from 650 cGy to 700 cGy. In some embodiments, the RBS delivers a radiation dose from 700 cGy to 750 cGy. In some embodiments, the RBS delivers a radiation dose from 750 cGy to 800 cGy. In some embodiments, the RBS delivers a radiation dose from 800 cGy to 850 cGy. In some embodiments, the RBS delivers a radiation dose from 850 cGy to 900 cGy. In some embodiments, the RBS delivers a radiation dose from 900 cGy to 950 cGy. In some embodiments, the RBS delivers a radiation dose from 950 cGy to 1000 cGy. In some embodiments, the RBS delivers a radiation dose from 1000 cGy to 1050 cGy. In some embodiments, the RBS delivers a radiation dose from 1050 cGy to 1100 cGy. In some embodiments, the RBS delivers a radiation dose from 1100 cGy to 1150 cGy. In some embodiments, the RBS delivers a radiation dose from 1150 cGy to 1200 cGy. In some embodiments, the RBS delivers a radiation dose from 1200 cGy to 1250 cGy. In some embodiments, the RBS delivers a radiation dose from 1250 cGy to 1300 cGy. In some embodiments, the RBS delivers a radiation dose from 1300 cGy to 1350 cGy. In some embodiments, the RBS delivers a radiation dose from 1350 cGy to 1400 cGy. In some embodiments, the RBS delivers a radiation dose from 1400 cGy to 1450 cGy. In some embodiments, the RBS delivers a radiation dose from 1450 cGy to 1500 cGy. In some embodiments, the RBS delivers a radiation dose from 1500 cGy to 1550 cGy. In some embodiments, the RBS delivers a radiation dose from 1550 cGy to 1600 cGy. In some embodiments, the RBS delivers a radiation dose from 1600 cGy to 1800 cGy. In some embodiments, the RBS delivers a radiation dose from 1800 cGy to 2000 cGy. In some embodiments, the RBS delivers a radiation dose of 600, 650, 700, 750, 800, 850, 900, 950, 1000, 1050, 1100, 1150, 1200, 1250, 1300, 1350, 1400, 1450, or 1500 cGy to the target. In some embodiments, the RBS delivers a radiation dose of 1500 to 3200 cGy. In some embodiments, the RBS delivers a radiation dose of 3200 to 8000 cGy. In some embodiments, the RBS delivers a radiation dose of 8000 cGy to 10000 cGy. In some embodiments, the RBS delivers a radiation dose of greater than 10000 cGy.

In some embodiments, the RBS delivers the prescribed dose in a time from 10 seconds to 20 minutes. In some embodiments, the RBS delivers the prescribed dose in a time from 20 seconds to 10 minutes. In some embodiments, the RBS delivers the prescribed dose in a time from 20 seconds to 60 seconds. In some embodiments, the RBS delivers the prescribed dose in a time from 30 seconds to 90 seconds. In some embodiments, the RBS delivers the prescribed dose in a time from 60 seconds to 90 seconds. In some embodiments, the RBS delivers the prescribed dose in a time from 90 seconds to 2 minutes. In some embodiments, the RBS delivers the prescribed dose in a time from 2 minutes to 3 minutes.

In some embodiments, the RBS delivers the prescribed dose in a time from 3 minutes to 4 minutes. In some embodiments, the RBS delivers the prescribed dose in a time from 3 minutes to 5 minutes. In some embodiments, the RBS delivers the prescribed dose in a time from 3 minutes to 6 minutes. In some embodiments, the RBS delivers the prescribed dose in a time from 4 minutes to 5 minutes. In some embodiments, the RBS delivers the prescribed dose in a time from 4 minutes to 6 minutes. In some embodiments, the RBS delivers the prescribed dose in a time from 5 minutes to 6 minutes. In some embodiments, the RBS delivers the prescribed dose in a time from 6 minutes to 7 minutes. In some embodiments, the RBS delivers the prescribed dose in a time from 7 minutes to 8 minutes. In some embodiments, the RBS delivers the prescribed dose in a time from 8 minutes to 9 minutes. In some embodiments, the RBS delivers the prescribed dose in a time from 9 minutes to 10 minutes. In some embodiments, the RBS delivers the prescribed dose in a time from 10 minutes to 12 minutes. In some embodiments, the RBS delivers the prescribed dose in a time from 12 minutes to 15 minutes. In some embodiments, the RBS delivers the prescribed dose in a time from 15 minutes to 20 minutes.

In some embodiments, the RBS delivers the prescribed dose in 25 seconds. In some embodiments, the RBS delivers the prescribed dose in 45 seconds. In some embodiments, the RBS delivers the prescribed dose in 60 seconds. In some embodiments, the RBS delivers the prescribed dose in 90 seconds. In some embodiments, the RBS delivers the prescribed dose in 2 minutes. In some embodiments, the RBS delivers the prescribed dose in 3 minutes. In some embodiments, the RBS delivers the prescribed dose in 4 minutes. In some embodiments, the RBS delivers the prescribed dose in 5 minutes. In some embodiments, the RBS delivers the prescribed dose in 6 minutes. In some embodiments, the RBS delivers the prescribed dose in 7 minutes. In some embodiments, the RBS delivers the prescribed dose in 8 minutes. In some embodiments, the RBS delivers the prescribed dose in 9 minutes. In some embodiments, the RBS delivers the prescribed dose in 10 minutes. In some embodiments, the RBS delivers the prescribed dose in 11 minutes. In some embodiments, the RBS delivers the prescribed dose in 12 minutes. In some embodiments, the RBS delivers the prescribed dose in 13 minutes. In some embodiments, the RBS delivers the prescribed dose in 14 minutes. In some embodiments, the RBS delivers the prescribed dose in 15 minutes. In some embodiments, the RBS delivers the prescribed dose in 16 minutes. In some embodiments, the RBS delivers the prescribed dose in 17 minutes. In some embodiments, the RBS delivers the prescribed dose in 18 minutes. In some embodiments, the RBS delivers the prescribed dose in 19 minutes. In some embodiments, the RBS delivers the prescribed dose in 20 minutes. In some embodiments, the RBS delivers the prescribed dose in a time frame greater than 20 minutes.

In some embodiments, a dose (e.g., a prescribed dose) may be delivered in a single application. In other embodiments, a dose (e.g., a prescribed dose) may be fractionated and applied in multiple applications. For example, in some embodiments, radiation (e.g., a prescribed dose) may be applied over the course of 2 applications. In some embodiments, radiation (e.g., a prescribed dose) may be applied over the course of 3 applications. In some embodiments, radiation (e.g., a prescribed dose) may be applied over the course of 4 applications. In some embodiments, radiation (e.g., a prescribed dose) may be applied over the course of 5 applications. In some embodiments, radiation (e.g., a prescribed dose) may be applied over the course of more than 5 applications. In some embodiments, radiation (e.g., a prescribed dose) may be applied over the course of 20 applications. In some embodiments, radiation (e.g., a prescribed dose) may be applied over the course of more than 20 applications.

Each application may deliver an equal sub-dose. In some embodiments, one or more of the sub-doses are different. For example, one or more of the sub-doses may be different so as to increase or decrease with each additional application.

According to one embodiment, a dose of radiation may be applied prior to the procedure for implantation of a MIGS device. For example, in some embodiments, a dose of radiation may be applied one or more days prior to the MIGS implantation surgery (e.g., insertion of the MIGS device). In some embodiments, a dose of radiation may be applied within a 24-hour prior before the MIGS implantation surgery (e.g., insertion of the MIGS device). In some embodiments, a dose of radiation may be applied just prior to the MIGS implantation surgery (e.g., insertion of the MIGS device), e.g., 1 hour before, 30 minutes before, 15 minutes before, 5 minutes before 1 minute before, etc. In some embodiments, a dose of radiation may be applied during the procedure for implantation of a MIGS device. In some embodiments, a dose of radiation may be applied right after the MIGS device is implanted, e.g., within 1 minute, 2 minutes, 3 minutes, 5 minutes, 10 minutes, etc.). In some embodiments, a dose of radiation may be applied before an incision is made into the conjunctiva. In some embodiments, a dose of radiation may be applied after an incision is made into the conjunctiva. In other embodiments, a dose of radiation may be applied after the MIGS implantation surgery (e.g., insertion of the MIGS device). In some embodiments, a dose of radiation may be applied within a 24-hour period after the MIGS implantation surgery (e.g., insertion of the MIGS device). In some embodiments, a dose of radiation may be applied within one to two days after the MIGS implantation surgery (e.g., insertion of the MIGS device). In some embodiments, a dose of radiation may be applied within 2 or more days after the MIGS implantation surgery (e.g., insertion of the MIGS device). In some embodiments the dose may be applied any time after the glaucoma surgery. In some embodiments, the dose is applied months or years after the glaucoma surgery. For example, a dose may be given to patients that did not receive a dose during surgery but at a future date have scar or needling procedures to break up scar tissue.

The present invention also provides applicators for applying the beta radiation to the target in the eye. In certain embodiments, the applicator may feature the RBS fixedly attached to the applicator. In some embodiments, the RBS is loaded in the applicator prior to its use in surgery. Devices may be similar to these originally used for pterygium or other ophthalmic applications. For example, the Technical Information and Instruction Manual for Users of the Beta Therapy Source Model 67-850, Nuclear Associates Manual lists multiple ophthalmic brachytherapy indications for use including: tumors, hemangioma, pterygium, vascularization, and irritable scar. However, the present invention is not limited to these previously made devices.

The applicator may be constructed from any appropriate material, such as a biocompatible material or a combination of materials. Non-limiting examples of biocompatible materials include, but are not limited to, metals (for example, stainless steel, titanium, gold), ceramics and polymers.

The applicator may comprise a handle adapted to hold the RBS, e.g., the RBS may be positioned at a distal end of the handle. In some embodiments, the applicator of the present invention comprises a radiation attenuation mask for shaping the radiation in a particular manner. For example, the mask may limit the amount of radiation that reaches non-target tissues such as the lens.

In some embodiments, the applicator features a removable cap for temporarily shielding the RBS or for keeping the applicator or RBS sterile.

In some embodiments, one or more components of the invention (e.g., applicator) are constructed from a material that can further shield the user from the RBS. In some embodiments, a material having a low atomic number (Z) may be used for shielding (e.g., polymethyl methacrylate). In some embodiments, one or more layers of material are used for shielding, wherein an inner layer comprises a material having a low atomic number (e.g., polymethyl methacrylate) and an outer layer comprises lead.

As an example, in some embodiments, the present invention is a device loaded from a Ruthenium-106 cow with an activity of rhodium-106 providing for the prescribed dose. The device can be applied to the target volume to deliver the full activity of its contents. For example, the device may be placed over the target lesion for 10 half-lives (300 seconds), delivering all its radioactive energy and consuming the rhodium-106, depleting it to palladium.

As an example, in some embodiments, the present invention is an applicator constructed containing Strontinum-90/Yttrium-90 radioisotopes in secular equilibrium. In some embodiments, the Sr-90/Y-90 is in a sealed source brachytherapy device, e.g., constructed of stainless steel. The source may be constructed to project a dose of about 1,000 cGy per unit time into a sufficient portion of the adjacent Planning Treatment Volume, e.g., to contain the conjunctival tissue to a depth of 0.3 mm. The source may be secured to a handle, and a radiation attenuation mask (shaped as a fan) is fixed to the source. The source may be covered with a sterile barrier. The present invention is not limited to this embodiment, and variations and combinations of the disclosed features are also covered in the scope of this application.

As previously discussed, the present invention provides methods for applying beta radiation to a target of the eye, for example the site of a bleb formed by a MIGS implant or procedure. Without wishing to limit the present invention to any theory or mechanism, it is believed that the use of beta radiation to treat the site of the bleb is advantageous because the application of beta radiation can be rapid and simple, and the effects can be long lasting. Further, beta radiation may be advantageous since it does not require post-operative compliance.

In some embodiments, the methods herein inhibit or reduce fibrogenesis in a bleb associated with a MIGS implant or procedure. In some embodiments, the methods herein inhibit or reduce inflammation in a bleb associated with a MIGS implant or procedure.

In some embodiments, the methods herein maintain the function of a bleb associated with a MIGS implant or procedure. In some embodiments, the methods herein enhance the function of a MIGS implant, e.g., by maintaining a functional bleb. In some embodiments, the methods herein reduce intraocular pressure (IOP), maintain a healthy IOP, treat glaucoma, etc.

The methods herein may comprise implanting a Minimally Invasive Glaucoma Surgery (MIGS) implant within the eye. MIGS implants are discussed in detail above. Generally, the MIGS implant is inserted trans-sclerally and causes formation of a bleb in the subconjunctival space of the eye or in a space between the conjunctiva and Tenon's capsule. For example, the MIGS implant may be placed between the anterior chamber of the eye and a subconjunctival space. In some embodiments, the MIGS implant is placed between the anterior chamber of the eye and a space between the conjunctiva and Tenon's capsule.

The methods herein comprise applying beta radiation to a target area of the eye. In some embodiments, the target area is a site of the bleb or an expected site of the bleb. In some embodiments, the target area surrounds the end of the implant. In some embodiments, the target is from 2 to 5 mm in diameter. In some embodiments, the target is from 5 to 12 mm in diameter. In some embodiments, the target is from 0.3 mm to 0.5 mm in thickness.

In some embodiments, the beta radiation is applied prior to the insertion of the MIGS implant. In some embodiments, the beta radiation is applied after the insertion of the MIGS implant.

In some embodiments, the methods herein further comprise introducing a drug to a site, e.g., a site of the MIGS implant, a site of the bleb, a different part of the eye. In certain embodiments, the drug is an antimetabolite. In certain embodiments, the drug is an anti-angiogenesis compound. In some embodiments, the drug is an anti-VEGF compound. As used herein, the term VEGF may refer to any appropriate VEGF, e.g., VEGF-1A, VEGF-1B, VEGF-1C, VEGF-1D, VEGF-1E, or other VEGF molecules or equivalents. As used herein, the term “anti-VEGF” compound or composition may refer to any compound, composition, molecule, etc. that inhibits angiogenesis, or inhibits VEGF or components in VEGF pathways (e.g., receptors, signaling molecules) that thus inhibit angiogenesis. Thus, “anti-VEGF” may be interchanged with “anti-angiogenesis.” Non-limiting examples of anti-VEGF (or equivalent) compounds, molecules, or compositions include antibodies, small molecules, drugs, or any appropriate composition for achieving effective anti-VEGF properties. Non-limiting examples of anti-VEGF/antiogenesis compounds or compositions include pegaptanib, bevacizumab, ranibizumab, axitinib, cabozantinib, everolimus, lenalidomide, lenvatinib mesylate, pazopanib, ramucirumab, regorafenib, sorafenib, sunitinib, thalidomide, vandetanib, ziv-aflibercept, or future anti-angiogenesis or anti-VEGF or anti-VEGF pathway molecules.

As previously discussed, ionizing radiation has effects on cells that can lead to cell cycle arrest. In some embodiments, the beta radiation of the present invention causes cell cycle arrest in fibroblasts on or associated with the Tenon's capsule or conjunctiva so as to inhibit or reduce the fibrotic process and inflammation that leads to bleb failure.

As previously discussed, the beta radiation may be applied via a radionuclide brachytherapy source (RBS). The RBS may be applied to the target via an applicator. As previously discussed, in some embodiments, the beta radiation is Strontium-90 (Sr-90), Phosphorus-32 (P-32), Ruthenium 106 (Ru-106), Yttrium 90 (Y-90), or a combination thereof. As previously discussed, in some embodiments, the RBS provides a dose of about 750 cGy to the target. In some embodiments, the RBS provides a dose from 500 to 1000 cGy to the target.

The present invention also features methods for preparing an applicator for emitting beta radiation. In some embodiments, the method comprises inserting a radionuclide brachytherapy source (RBS) into a RBS cavity in an applicator. In some embodiments, the method comprises attaching the RBS to an applicator. In some embodiments, the applicator comprises a handle and a distal portion, wherein the distal portion is where the RBS is attached or is the site of the RBS cavity. In some embodiments, the RBS is constructed to emit a radiation dose at 4 mm from its center that is at least 90% of that emitted at the center. The present invention is not limited to an RBS emitting a radiation dose at 4 mm from its center that is at least 90% of that emitted at the center. Alternative examples of dose distributions are described herein.

### Example 1: Surgical Procedure for Beta Radiation Application

The present invention provides an example of a procedure for the application of beta radiation to the eye. The present invention is in no way limited to the specific steps, methods, devices, systems, and compositions described herein.

### Preparation and Assembly

The device assembly procedure may be done behind a plexiglass beta shield (for example, the Large Dual Angle Beta Radiation Shield, Universal Medical Inc.). The medical technician or medical physicist or other user opens the Radioisotope Brachytherapy Source (RBS) storage container. The RBS is removed from its container using appropriate handling techniques (for example, long forceps). The RBS is placed on a clean field.

The Brachytherapy Applicator may be a single-use sterile-packed device. Its packaging may be checked by examining for damage or breach of the sterile barrier. If finding none, the Brachytherapy Applicator package is opened, and the applicator assembly placed on a sterile field.

The Brachytherapy Applicator comprises a handle and an RBS cap. Using aseptic technique and remote handling techniques, the RBS is loaded into the Brachytherapy Applicator, e.g., the RBS may be inserted into the cap and the handle is subsequently connected to the cap, securing the RBS. Care is taken to avoid contamination.

The radiation output may be confirmed consistent with standards of quality assurance in radiation therapy (for example see: Palmer, Antony L., Andrew Nisbet, and David Bradley. “Verification of high dose rate brachytherapy dose distributions with EBT3 Gafchromic film quality control techniques.” Physics in medicine and biology 58.3 (2013): 497). In one method of quality assurance, the applicator is applied to radiographic film in sterile overwrap for a specified dwell time (for example Gafchromic® film, Ashland Inc.). The overwrap is removed. The medical physicist checks the area of application for evidence of film exposure.

The device may be placed into a sterile plexiglass beta transport box (for example the IBI Beta-Gard Acrylic Storage Container—Large, Universal Medical Inc.) and the box placed on the operative Mayo stand.

Previously the decayed activity of the RBS has been calculated to determine the contemporary dose per unit time (for example, cGy/second). The decay calculation methodology is known to those skilled in medical physics and is also described in the NRC Information Notice 96-66: United States Nuclear Regulatory Commission, Office of Nuclear Material Safety and Safeguards, Washington D.C. 20555, Dec. 13, 1996. The dwell time for the total prescribed dose is then calculated. As an example, the prescription dose is 1,000 cGy to a center point of 0.19 mm depth from the conjunctival surface. As an example, the decayed activity of the RBS is 30 cGy/second at a water equivalent depth of 0.19 mm. In this example, the dwell time is calculated to be about 33 seconds, providing a 990 cGy dose.

### Surgical Application

The beta therapy may be applied following completion of a glaucoma surgery. (Note the present invention is not limited to applying beta radiation after glaucoma surgery.) The eye is rotated to a downward gaze position by the use of a probe placed against the sclera providing traction (for example the distal end of a Vera Hook placed against the eye). This allows better visual and surgical access to the superior conjunctiva.

The ophthalmic surgeon obtains the Brachytherapy Applicator device, e.g., from the transport box. The tip (e.g., distal end, active end) of the applicator is placed over the conjunctiva in a position just superior to the limbus. The diameter of the applicator encompasses the appropriate surface area of the target, e.g., bleb. The Brachytherapy Applicator is pressed to the surface of the eye. In some embodiments, the Brachytherapy Applicator is pressed to the surface of the eye such that all or substantially all of the edema fluid is pushed away. The Applicator is held in place for the specified dwell time. In some embodiments, the dwell time has been programmed into a count-down clock. Following the specified dwell time, the Brachytherapy Applicator is removed from the operative field.

At the conclusion of surgery, antibiotic ointment is applied to the eye and the eye patched.

In certain embodiments, following the surgery, the Brachytherapy Applicator is disassembled behind the acrylic beta shield. The Radioisotope Brachytherapy Source is returned to its storage container. The disposable portions of the device are discarded in a manner consistent with appropriate disposal of biological waste (for example “red bag” waste).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

Any reference numbers recited herein, including the claims below, are solely for ease of examination of this patent application, and are exemplary, and are not intended in any way to limit the scope of the claims to the particular features having the corresponding reference numbers in the drawings.

What is claimed is:

1. A method of maintaining a functioning drainage bleb in the eye of a patient being treated for glaucoma, the method comprising:
applying a therapeutic dose of beta radiation from a radioisotope to a target area of the eye using an applicator system, the target area is at least a portion of the bleb, the applicator system comprises a handle and a distal end with the radioisotope embedded or engaged therein, the distal end has an outer surface for contacting the eye, wherein the outer surface of the distal end of the applicator system is placed in contact with the eye and pressed upon such that at least a portion of conjunctiva edema fluid is pushed away;
wherein the therapeutic dose of beta radiation reduces or inhibits a fibrotic process and inflammation that causes bleb failure, and wherein the method is effective to maintain the drainage function of the bleb.

2. The method of claim 1, wherein a Minimally Invasive Glaucoma Surgery (MIGS) implant is inserted trans-sclerally.

3. The method of claim 1, wherein the distance from the outer surface of the distal end of the applicator system and the bottom surface of the bleb is substantially uniform across the target area.

4. The method of claim 1, wherein the outer surface of the distal end of the applicator system is flat.

5. The method of claim 1, wherein the outer surface of the distal end of the applicator system has curvature.

6. The method of claim 1, wherein the outer surface has a portion that has curvature and a portion that is flat.

7. The method of claim 1, wherein the outer surface of the distal end is from 8 to 10 mm in diameter.

8. The method of claim 1, wherein the outer surface of the distal end is from 10 to 12 mm in diameter.

9. The method of claim 1, wherein at least 50% of the surface area of the outer surface of the distal end is in contact with the eye.

10. The method of claim 1, wherein the target area surrounds an end of a MIGS implant.

11. The method of claim 1, wherein the radioisotope comprises Strontium-90 (Sr-90), Phosphorus-32 (P-32), Ruthenium 106 (Ru-106), Yttrium 90 (Y-90), or a combination thereof.

12. The method of claim 1, wherein the therapeutic dose of beta radiation is from 250-1000 cGy.

13. The method of claim 1, wherein the therapeutic dose of beta radiation is from 500-3200 cGy.

14. The method of claim 1, wherein the method further comprises administering a drug to the target area.

15. The method of claim 14, wherein the drug is an antimetabolite.

16. The method of claim 1, wherein the method is effective for reducing IOP to 12 mmHg or less.

17. The method of claim 1, wherein the method is effective for reducing IOP by 20% or more 6 months after treatment.

18. The method of claim 1, wherein the method is effective for reducing IOP by 20% or more 12 months after treatment.

19. The method of claim 1, wherein the method is effective for reducing IOP and subsequent stabilization of said IOP.

20. The method of claim 19, wherein stabilization of IOP is wherein the IOP does not increase by more than 20% at 6 months after treatment.
